SEQUENCE LISTING (6 sequences)

SEQ ID NO: 1 - protein, 1250 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 2 - protein, 191 aa, Homo sapiens [sequence omitted]

SEQ ID NO: 3 - protein, 1104 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - protein, 1250 aa, Artificial Sequence; identical to SEQ ID NO: 1
  Description of Artificial Sequence Synthetic polypeptide

SEQ ID NO: 5 - protein, 1394 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 6 - protein, 34 aa, Artificial Sequence
  Description of Artificial Sequence Synthetic polypeptide
PATSGSETPGTSESATPESGPGTSTEPSEGSAPG

(19) United States
(12) Patent Application Publication
(10) Pub. No.: US 2020/0000884 A1
(43) Pub. Date: Jan. 2, 2020

(54) TREATMENT OF ADULT GROWTH HORMONE DEFICIENCY WITH HUMAN GROWTH HORMONE ANALOGUES

(71) Applicant: AMUNIX PHARMACEUTICALS, INC., Mountain View, CA (US)

(72) Inventors: Daniela ROGOFF, Menlo Park, CA (US); Eric HUMPHRISS, Menlo Park, CA (US); Will CHARLTON, Menlo Park, CA (US); Bert BAKKER, Menlo Park, CA (US); Shelby YOUNG, Menlo Park, CA (US)

(21) Appl. No.: 16/320,830

(22) PCT Filed: Jul. 27, 2017
(86) PCT No.: PCT/US17/44251

§ 371 Date: Jan. 25, 2019

Related U.S. Application Data
(60) Provisional application No. 62/367,476, filed on Jul. 27, 2016.
(60) Provisional application No. 62/463,436, filed on Feb. 24, 2017.

(51) Int. Cl. A61K 38/27; A61K 9/00

(52) U.S. Cl. CPC A61K 38/27; A61K 9/0019

(57) ABSTRACT

The present disclosure provides a growth hormone deficiency (GHD) therapy for adult subjects.

### SEQUENCE LISTING

The instant application contains a Sequence Listing which has been filed electronically in ASCII format and is hereby incorporated by reference in its entirety. Said ASCII copy, created on Mar. 13, 2019, is named 32808-764_831_SL.txt and is 46,091 bytes in size.

### BACKGROUND OF THE INVENTION

Growth Hormone Deficiency (GHD) occurs in children and adults, and results from a variety of genetic, neoplastic, inflammatory, traumatic and iatrogenic causes. The consequences of GHD will depend on the severity of the deficiency and the age at which the deficiency occurs. Subjects with untreated childhood onset GHD will have significant growth failure with attainment of adult heights significantly less than five feet in many instances. In addition, there is abnormal body composition with decreased bone mineralization, decreased lean body mass and increased fat mass. In children, treatment with exogenous recombinant growth hormone (rhGH) promotes normal body composition and initiates a period of accelerated or “catchup” growth that when begun at an early age allows attainment of normal adult height. GHD is also a well-recognized clinical syndrome in adults. In the adult, GHD may be a continuation of the childhood-onset condition or result from damage to the pituitary during adult life. Manifestations of the adult GHD syndrome include altered body composition (decreased muscle mass, increased fat mass, osteopenia), alterations in glucose and lipid metabolism leading to increased risk for adverse cardiovascular events, decreased exercise capacity and decreased quality of life.

There are currently no preventative treatments for GHD in children or adults. Growth hormone (GH) replacement therapy provides positive effects on body composition, lipid metabolism, bone metabolism, and quality of life in adults with GHD. Daily subcutaneous dosing of rhGH is the currently approved therapy for the replacement of insufficient endogenous secretion of GH. Dosing of rhGH is typically based on the body weight of the patient. Daily injections can be a challenge, and a lack of compliance with daily rhGH administration is commonplace and can lead to loss of treatment effects. Additionally, many adult GHD patients choose not to initiate therapy due to the onus of daily injections, thereby missing the opportunity to mitigate the cardiometabolic risks associated with GHD. Long-acting rhGH offers the possibilities of fewer injections, enhanced compliance and attainment of improved treatment outcomes. Thus, the lack of compliance is an important barrier to achieve the benefits of the treatment.

Thus, there is a need to develop alternative treatment in GHD adults to provide an alternative to daily dosing that may avoid these compliance and adherence problems and attendant loss of treatment effect. VRS-317 (SEQ ID NO:1, FIG. 1) is a novel fusion protein (M.W. 119 kDa) designed to improve upon currently approved growth hormone drugs and consists of rhGH with amino acid sequences (XTEN) attached at the N- and C-termini. VRS-317 is designed to maintain active drug levels for a longer period of time than currently available therapies. Compositions and methods related to VRS-317 are described in, for example, U.S. Pat. No. 8,703,717, U.S. Patent Publication No. 2014-0162949, and WO/2014/164568 (U.S. Patent Publication No. 2016-0158321), which are incorporated herein by reference in their entirety. In some embodiments, the novel fusion protein designed to improve upon currently approved growth hormone drugs retains the biological activity of VRS-317 and comprises an amino acid sequence having at least about 80%, or alternatively, at least about 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1.

### SUMMARY OF THE INVENTION

The present disclosure provides methods, compositions, apparatuses and kits for a growth hormone deficiency (GHD) therapy.

Aspects and embodiments of the compositions, kits, apparatuses and methods are set forth in the following paragraphs.

In one embodiment, the present disclosure provides a method of treating human adult growth hormone deficiency (AGHD) in an adult patient, the treatment comprising administering to the adult patient with AGHD a human growth hormone-XTEN (hGH-XTEN) fusion protein at a dose between about 5 mg/administration and about 500 mg/administration, wherein the dose is not based on a body weight of said adult patient. In another embodiment, the administering is at a frequency of twice a month (15 days+/−2 days). In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, and the N-terminus XTEN domain of SEQ ID NO: 1. In another embodiment, the treatment continues for at least about 6 months from first administration. In another embodiment, the treatment continues for at least about 12 months from first administration. In another embodiment, the first and the second XTEN are sequences that exhibit at least about 90% sequence identity to a sequence selected from the C-terminus XTEN domain of SEQ ID NO: 1, and the N-terminus XTEN domain of SEQ ID NO: 1. In another embodiment, the dose of the hGH-XTEN fusion protein is administered subcutaneously. In another embodiment, the dose of hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration. In another embodiment, the patient has a serum IGF-I standard deviation score (SDS) between about −2.0 and about 2.0 following administration. In another the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In another embodiment, the hGH-XTEN fusion protein comprises the amino acid sequence of SEQ ID NO:1. In another embodiment, the dose is effective to maintain a plasma concentration of said fusion protein in the patient at more than about 10 ng/mL for a period of at least 10 days after administration. In another embodiment, the AGHD patient is a woman on estrogen therapy and the dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration. In another embodiment, the AGHD patient is not a woman on estrogen therapy and the dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration. In another embodiment, the AGHD patient is a patient having a baseline IGF-I SDS of less or equal than −1 and the dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration. In another embodiment, the AGHD patient is a patient having a baseline IGF-I SDS of higher than −1 and the dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

In one embodiment, the present disclosure provides a method of treating AGHD in an adult patient, the treatment comprising: (a) administering to the adult patient with AGHD a hGH-XTEN fusion protein at a dose selected from (i) 20 mg for all subjects other than women receiving oral estrogen, or (ii) 40 mg for women receiving oral estrogen; (b) monitoring the IGF-I standard deviation score (SDS) in a plasma or serum sample obtained from the patient during an initial dosage period of administration of an initial dose of hGH-XTEN fusion protein; and (c) determining a subsequent dose of hGH-XTEN fusion protein administered over a subsequent dosage period based on the IGF-I SDS observed during the initial dosage period; wherein the dose is not based on a body weight of said adult patient. In another embodiment, the administering is at a frequency of twice a month (15 days+/−2 days). In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, and the N-terminus XTEN domain of SEQ ID NO: 1. In another embodiment, the hGH-XTEN fusion protein comprises the amino acid sequence of SEQ ID NO:1. In another embodiment, the method comprises administering a plurality of subsequent doses between 5 mg/administration and 500 mg/administration until a maintenance dose is achieved. In another embodiment, the maintenance dose is effective to maintain the patient's serum IGF-I standard deviation score (SDS) between −2.0 and 2.0 SDS for 7 days after two consecutive subsequent dose administrations. In another embodiment, the maintenance dose is effective to maintain the patient's serum IGF-I standard deviation score (SDS) between −1.5 and 2.0 SDS for 7 days after two consecutive subsequent dose administrations. In another embodiment, the maintenance dose is effective to maintain the patient's serum IGF-I standard deviation score (SDS) between 0 and 2.0 SDS for 7 days after two consecutive subsequent dose administrations. In another embodiment, the dose of the hGH-XTEN fusion protein is administered subcutaneously. In another embodiment, the dose of hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration. In another embodiment, the maintenance dose is effective to maintain a plasma concentration of said fusion protein in the patient at more than about 10 ng/mL for a period of at least 10 days after administration. In another embodiment, the AGHD patient is a woman on estrogen therapy and the dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration. In another embodiment, the AGHD patient is not a woman on estrogen therapy and the dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration. In another embodiment, the AGHD patient is a patient having a baseline IGF-I SDS of less or equal than −1 and the dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration. In another embodiment, the AGHD patient is a patient having a baseline IGF-I SDS of higher than −1 and the dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

In one embodiment, the present disclosure provides a method for the treatment of AGHD in an adult patient comprising: administering to an adult with AGHD a hGH-XTEN fusion protein at an initial dose between about 5 mg/administration and about 500 mg/administration, and as a plurality of subsequent doses between about 5 mg/administration and about 500 mg/administration, wherein the initial dose and the plurality of subsequent doses are not based on a body weight of said adult patient. In another embodiment, administering is at a frequency of twice a month (15 days+/−2 days). In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, and the N-terminus XTEN domain of SEQ ID NO: 1. In another embodiment, the plurality of subsequent doses are effective to maintain the patient's serum IGF-I standard deviation score (SDS) between −2.0 and 2.0 SDS for 7 days after administration. In another embodiment, the plurality of subsequent doses are effective to maintain the patient's serum IGF-I standard deviation score (SDS) between −1.5 and 2.0 SDS for 7 days after administration. In another embodiment, the plurality of subsequent doses are effective to maintain the patient's serum IGF-I standard deviation score (SDS) between 0 and 2.0 SDS for 7 days after administration. In another embodiment, the dose of the hGH-XTEN fusion protein is administered subcutaneously. In another embodiment, either the initial dose or any of said plurality of doses of said hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration. In another embodiment, the plurality of subsequent doses are effective to maintain a plasma concentration of said fusion protein in the patient at more than about 10 ng/mL for a period of at least 10 days after administration. In another embodiment, the AGHD patient is a woman on estrogen therapy, and wherein either the initial dose or any of the plurality of doses of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration. In another embodiment, the AGHD patient is not a woman on estrogen therapy, and wherein either the initial dose or any of the plurality of doses of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration. In another embodiment, the AGHD patient is a patient having a baseline IGF-I SDS of less or equal than −1, and wherein either the initial dose or any of the plurality of doses of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration. In another embodiment, the AGHD patient is a patient having a baseline IGF-I SDS of higher than −1, and wherein either the initial dose or any of the plurality of doses of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

In one embodiment, the present disclosure provides a method of treating AGHD in an adult patient, the treatment comprising: (a) administering to the adult patient with AGHD an hGH-XTEN fusion protein at an initial dose selected from (i) 20 mg for all subjects other than women receiving oral estrogen, or (ii) 40 mg for women receiving oral estrogen; (b) decreasing or increasing one or more subsequent dosage amounts of the hGH-XTEN fusion protein until a maintenance dose is achieved, wherein said maintenance dose is achieved when the patient's serum IGF-I SDS is between about −2.0 and about 2.0 for two consecutive 7 post-dose days after administration; (c) administering a plurality of subsequent maintenance doses; wherein the initial dose, the one or more subsequent doses, and the plurality of subsequent maintenance doses are not based on a body weight of said adult patient. In another embodiment, the administering is at frequency of twice a month (15 days+/−2 days). In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, and the N-terminus XTEN domain of SEQ ID NO: 1. In another embodiment, the maintenance dose is effective to maintain the patient's serum IGF-I SDS between about −2.0 and about 2.0 for (i) at least 7 days; (ii) at least about 10 days; or (iii) at least about 20 days after administration. In another embodiment, the hGH-XTEN fusion protein comprises the amino acid sequence of SEQ ID NO:1. In another embodiment, the AGHD patient is a woman on estrogen therapy and the maintenance dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration. In another embodiment, the AGHD patient is not a woman on estrogen therapy and the maintenance dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration. In another embodiment, the AGHD patient is a patient having a baseline IGF-I SDS of less or equal than −1 and the maintenance dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration. In another embodiment, the AGHD patient is a patient having a baseline IGF-I SDS of higher than −1 and the maintenance dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides the amino acid sequence for VRS-317 (SEQ ID NO:1), an hGH-XTEN fusion protein (hGH sequence is underlined and bold).

FIGS. 2a and 2b show the Mean IGF-I SDS during the dosing interval. FIG. 2a shows the Mean IGF-I SDS at different time points during each month for Cohort 1 and Cohort 2. The target IGF-I SDS range is shadowed in gray. FIG. 2b shows the Mean Change in IGF-I SDS from month 1, day 1 in Cohort 1 and Cohort 2.

FIG. 3 shows the Mean±SD IGF-I SDS for subjects on a maintenance dose. The target IGF-I SDS range is shadowed in gray.

### DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

A “fragment” of a protein as used herein is a truncated form of a native biologically active protein that retains at least a portion of the therapeutic and/or biological activity. A “variant” of a protein as used herein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity with the reference biologically active protein. As used herein, the term “biologically active protein moiety” includes proteins modified deliberately, as for example, by site directed mutagenesis, insertions, or accidentally through mutations.

“Conjugated”, “linked,” “fused,” and “fusion” are used interchangeably herein. These terms refer to the joining together of two or more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature).

“Recombinant” as applied to a polynucleotide means that the polynucleotide is the product of various combinations of in vitro cloning, restriction and/or ligation steps, and other procedures that result in a construct that can potentially be expressed in a host cell.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2nd ed., vol. 1-3, Cold Spring Harbor Press, Plainview N.Y.; specifically see volume 2 and chapter 9. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “t½” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t½”, “terminal half-life”, “elimination half-life” and “circulating half-life” are used interchangeably herein.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect”, as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the disclosure other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose”, as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. For example, such effect may entail increasing the height of said patient.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, an excipient, a stabilizer, or preservative.

The term “therapeutically effective dose regimen”, as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

### I). General Techniques

The practice of various embodiments of the present disclosure employs, unless otherwise indicated, conventional techniques of immunology, biochemistry, chemistry, molecular biology, microbiology, cell biology, genomics and recombinant DNA, which are within the skill of the art. See, for example, Sambrook, J. et al., “Molecular Cloning: A Laboratory Manual,” 3rd edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R. I., “Culture of Animal Cells: A Manual of Basic Technique,” 4th edition, John Wiley & Sons, Somerset, N J, 2000, the contents of which are incorporated in their entirety herein by reference.

### II). Growth Hormone

The present invention concerns an improved therapeutic regimen for human growth hormone deficiency (GHD) therapy. In particular, the invention concerns methods for administration of a human growth hormone-XTEN (hGH-XTEN) fusion protein to a patient with GHD. Accordingly, in one aspect, the present invention concerns a method of treating GHD with a hGH-XTEN fusion protein.

(a) Growth Hormone Proteins

“Growth Hormone” or “GH” means a growth hormone protein and species and sequence variants thereof, and includes, but is not limited to, the 191 single-chain amino acid sequence of human GH. The GH can be the native, full-length protein or can be a truncated fragment or a sequence variant that retains at least a portion of the biological activity of the native protein. There are two known types of human GH (hereinafter “hGH”) derived from the pituitary gland: one having a molecular weight of about 22,129 daltons (22 kD hGH) and the other having a molecular weight of about 20,000 daltons (20 kD hGH). The 20 kD HGH has an amino acid sequence that corresponds to that of 22 kD hGH consisting of 191 amino acids except that 15 amino acid residues from the 32nd to the 46th of 22 kD hGH are missing. Some reports have shown that the 20 kD hGH has been found to exhibit lower risks and higher activity than 22 kD hGH. The invention contemplates use of the 22 kD, the 20 kD hGH, as well as species and sequence variants and truncated fragments thereof as being appropriate for use as a fusion partner with XTEN disclosed herein for hGH-XTEN compositions. The cloned gene for hGH has been expressed in a secreted form in Escherichia coli (U.S. Pat. No. 4,898,830; Chang, C. N., et al., Gene 55:189 [1987]) and its DNA and amino acid sequence has been reported (Goeddel, et al. Nature, 281:544 [1979]); Gray, et al., Gene 39: 247[1985]).

The invention contemplates inclusion in the hGH-XTEN compositions sequences with homology to GH sequences, sequence fragments that are natural, such as from humans and non-natural sequence variants which retain at least a portion of the biologic activity or biological function of GH and/or that are useful for preventing, treating, mediating, or ameliorating a GH-related disease, deficiency, disorder or condition. In addition, native sequences homologous to human GH may be found by standard homology searching techniques, such as NCBI BLAST.

Effects of GH on the tissues of the body can generally be described as anabolic. Like most other protein hormones, native GH acts by interacting with a specific plasma membrane receptor, referred to as growth hormone receptor. GH acts on the liver and other tissues to stimulate production of IGF-I, which is responsible for the growth promoting effects of GH and also reflects the amount produced. IGF-I, in turn, has stimulatory effects on osteoblast and chondrocyte activity to promote bone growth. In one embodiment, the invention provides a hGH-XTEN that exhibits at least one of the properties of native GH hereinabove described herein.

In one embodiment, the GH incorporated into the subject compositions is a recombinant polypeptide with a sequence corresponding to a protein found in nature. In another embodiment, the GH is a sequence variant, fragment, homolog, or a mimetics of a natural sequence that retains at least a portion of the biological activity of the corresponding native GH. In one other embodiment, the GH is human GH comprising the following amino acid sequence:

(SEQ ID NO: 2)
FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF.

Any human GH sequences or homologous derivatives constructed by shuffling individual mutations between families that retain at least a portion of the biological activity of the native GH may be useful for the fusion proteins of this invention. GH that can be incorporated into a hGH-XTEN fusion protein can include a protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:2. In some embodiments, the invention comprise the use a hGH variant that is at least about 90%, or alternatively, at least about 91%, 92%, 93%, 94%, 95%, 96%, 97%, 98%, 99%, or 100% sequence identity to SEQ ID NO:2. In some embodiments, the invention comprise the use a hGH variant that is at least about 95%, or alternatively, at least about 96%, 97%, 98%, 99%, or 100% sequence identity to either the SEQ ID NO:2.

### III). Human Growth Hormone-XTEN Fusion Protein Compositions for Treating GHD

Human growth hormone (hGH) is naturally secreted from the human anterior pituitary as intermittent pulses lasting from minutes to hours, typically occurring during sleep. To promote anabolic and growth processes, hGH binds to the hGH receptor initiating signaling processes involving the STAT (signal transducer and activator of transcription), the MAPK (mitogen-activated protein kinase) and the PI3K (phosphoinositide-3 kinase) pathways. Insulin-like growth factor-I (IGF-I) gene expression is activated from hGH receptor signaling resulting in secretion of IGF-I into the circulation. Although hGH retains unique biological actions, IGF-I is the primary mediator for the growth promoting effects of hGH. As such, IGF-I also serves as the primary pharmacodynamic (PD) marker for response to rhGH administration. In the circulation, IGF-I forms a complex with insulin-like growth factor binding protein-3 (IGFBP-3) and the acid labile subunit (ALS). Both IGFBP-3 and ALS expression are also regulated by hGH receptor activation.

Daily rhGH therapy does not mimic the typical endogenous pulsatile release of GH in healthy individuals. However, daily injections of rhGH have been demonstrated for over 30 years to be a safe and effective therapy for treatment of GHD. Clinical studies of continuous infusion of rhGH with a pump demonstrate comparable metabolic effects, growth velocity, and IGF-I responses to those achieved with daily rhGH injections. A continuous infusion of rhGH has been compared to daily rhGH therapy in adult GHD patients for 6 months and in GHD children for 6 months. These studies indicated that the safety profile and effects on the IGF-I responses were not significantly different between patients treated with continuous subcutaneous infusion of rhGH or once daily subcutaneous rhGH therapy. Therefore, continuous, as well as daily, administration of rhGH, neither of which follow endogenous secretion patterns, appear to be safe and efficacious. There is no preventative therapy for GHD and no other treatment modalities are known to be effective.

The present invention concerns an improved therapeutic regimen for GHD therapy. In particular, the invention concerns methods for administration of hGH-XTEN fusion proteins to an adult patient with GHD. In one aspect, the hGH fusion proteins suitable for use in the present invention comprise a human growth hormone polypeptide and one or more XTEN sequences as described herein, and as disclosed in Schellenberger et al. WO10/144502A2, WO10/091122, and U.S. Pat. Nos. 8,673,860 and 8,703,717, which are incorporated herein by reference in their entirety.

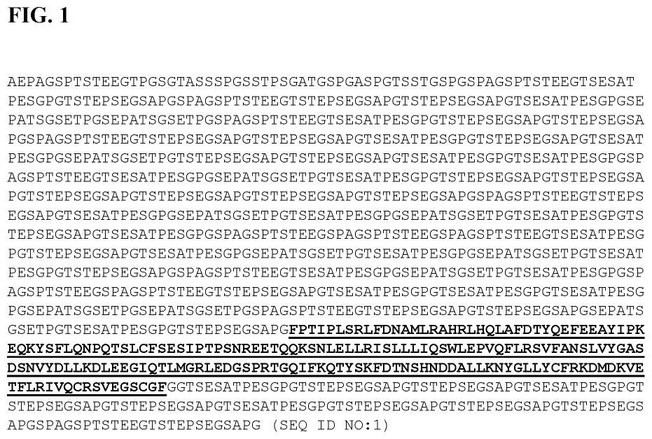

In one other aspect, the hGH-XTEN fusion proteins are fusion proteins of GH comprising the full-length sequence or sequence variants of GH covalently linked to one or more extended recombinant polypeptides (“XTEN” or “XTENs”). In one embodiment, the hGH-XTEN fusion protein comprises an amino acid sequence shown in FIG. 1 (SEQ ID NO: 1), or pharmacologically active variants thereof. In another embodiment, the hGH-XTEN fusion protein comprises an amino acid sequence selected from Table 1.

The fusion protein VRS-317, is composed of recombinant human growth hormone (rhGH) and two recombinant polypeptides, referred to as XTEN as described in Schellenberger et al. (2009). Nat Biotechnol 27, 1186-90, Schellenberger et al. WO10/144502A2, WO10/091122, and U.S. Pat. Nos. 8,673,860 and 8,703,717, each of which are incorporated herein by reference in their entirety. The XTEN domain, two unstructured hydrophilic chains of amino acids, provides half-life extension for rhGH. The molecular weight of VRS-317 is 118.9 kDa, with rhGH contributing 22.1 kDa and the remaining mass contributed by the XTEN construct. The mass ratio of rhGH to VRS-317 is therefore 1:5.37. The amino acid sequence of the VRS-317 fusion protein is provided in FIG. 1. In some embodiments, the novel fusion protein designed to improve upon currently approved growth hormone drugs retains at least a portion of the therapeutic and/or biological activity of VRS-317 and comprises an amino acid sequence having at least about 80%, or alternatively, at least about 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1.

Without intending to be limited to any theory, the N-terminus XTEN domain enables half-life extension of the rhGH by increasing the hydrodynamic diameter and molecular weight of rhGH, which reduces kidney filtration. An XTEN domain on the N-terminal, C-terminal, or both terminal ends of rhGH may also enable half-life extension of the rhGH by delaying receptor mediated clearance. VRS-317 is being developed as a long-acting alternative to daily rhGH injections. In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO: 2), and the first and/or the second XTEN are sequences that exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the C-terminus and the N-terminus XTEN domain of SEQ ID NO: 1.

TABLE 1
Exemplary hGH-XTEN fusion proteins

| hGH- | | |
|---|---|---|
| XTEN | | SEQ ID |
| Name* | Amino Acid Sequence | NO: |
| AE912. | AEPAGSPTSTEEGTPGSGTASSSPGSSTPSGATGSPGASPGTSSTGSP | 3 |
| 2-hGH | GSPAGSPTSTEEGTSESATPESGPGTSTEPSEGSAPGSPAGSPTSTEEG | |
| | TSTEPSEGSAPGTSTEPSEGSAPGTSESATPESGPGSEPATSGSETPGS | |
| | EPATSGSETPGSPAGSPTSTEEGTSESATPESGPGTSTEPSEGSAPGTS | |
| | TEPSEGSAPGSPAGSPTSTEEGTSTEPSEGSAPGTSTEPSEGSAPGTSE | |
| | SATPESGPGTSTEPSEGSAPGTSESATPESGPGSEPATSGSETPGTSTE | |
| | PSEGSAPGTSTEPSEGSAPGTSESATPESGPGTSESATPESGPGSPAGS | |
| | PTSTEEGTSESATPESGPGSEPATSGSETPGTSESATPESGPGTSTEPS | |
| | EGSAPGTSTEPSEGSAPGTSTEPSEGSAPGTSTEPSEGSAPGTSTEPSE | |
| | GSAPGTSTEPSEGSAPGSPAGSPTSTEEGTSTEPSEGSAPGTSESATPE | |
| | SGPGSEPATSGSETPGTSESATPESGPGSEPATSGSETPGTSESATPES | |
| | GPGTSTEPSEGSAPGTSESATPESGPGSPAGSPTSTEEGSPAGSPTSTE | |
| | EGSPAGSPTSTEEGTSESATPESGPGTSTEPSEGSAPGTSESATPESGP | |
| | GSEPATSGSETPGTSESATPESGPGSEPATSGSETPGTSESATPESGPG | |
| | TSTEPSEGSAPGSPAGSPTSTEEGTSESATPESGPGSEPATSGSETPGT | |
| | SESATPESGPGSPAGSPTSTEEGSPAGSPTSTEEGTSTEPSEGSAPGTS | |
| | ESATPESGPGTSESATPESGPGTSESATPESGPGSEPATSGSETPGSEP | |
| | ATSGSETPGSPAGSPTSTEEGTSTEPSEGSAPGTSTEPSEGSAPGSEPA | |
| | TSGSETPGTSESATPESGPGTSTEPSEGSAPGFPTIPLSRLFDNAMLRA | |
| | HRLHQLAFDTYQEFEEAYIPKEQKYSFLQNPQTSLCFSESIPTPSNRE | |
| | ETQQKSNLELLRISLLLIQSWLEPVQFLRSVFANSLVYGASDSNVYD | |
| | LLKDLEEGIQTLMGRLEDGSPRTGQIFKQTYSKFDTNSHNDDALLK | |
| | NYGLLYCFRKDMDKVETFLRIVQCRSVEGSCGF | |
| AE912. | AEPAGSPTSTEEGTPGSGTASSSPGSSTPSGATGSPGASPGTSSTGSP | 4 |
| 2-hGH- | GSPAGSPTSTEEGTSESATPESGPGTSTEPSEGSAPGSPAGSPTSTEEG | |
| AE146 | TSTEPSEGSAPGTSTEPSEGSAPGTSESATPESGPGSEPATSGSETPGS | |
| | EPATSGSETPGSPAGSPTSTEEGTSESATPESGPGTSTEPSEGSAPGTS | |
| | TEPSEGSAPGSPAGSPTSTEEGTSTEPSEGSAPGTSTEPSEGSAPGTSE | |
| | SATPESGPGTSTEPSEGSAPGTSESATPESGPGSEPATSGSETPGTSTE | |
| | PSEGSAPGTSTEPSEGSAPGTSESATPESGPGTSESATPESGPGSPAGS | |
| | PTSTEEGTSESATPESGPGSEPATSGSETPGTSESATPESGPGTSTEPS | |
| | EGSAPGTSTEPSEGSAPGTSTEPSEGSAPGTSTEPSEGSAPGTSTEPSE | |
| | GSAPGTSTEPSEGSAPGSPAGSPTSTEEGTSTEPSEGSAPGTSESATPE | |
| | SGPGSEPATSGSETPGTSESATPESGPGSEPATSGSETPGTSESATPES | |
| | GPGTSTEPSEGSAPGTSESATPESGPGSPAGSPTSTEEGSPAGSPTSTE | |
| | EGSPAGSPTSTEEGTSESATPESGPGTSTEPSEGSAPGTSESATPESGP | |
| | GSEPATSGSETPGTSESATPESGPGSEPATSGSETPGTSESATPESGPG | |
| | TSTEPSEGSAPGSPAGSPTSTEEGTSESATPESGPGSEPATSGSETPGT | |
| | SESATPESGPGSPAGSPTSTEEGSPAGSPTSTEEGTSTEPSEGSAPGTS | |
| | ESATPESGPGTSESATPESGPGTSESATPESGPGSEPATSGSETPGSEP | |
| | ATSGSETPGSPAGSPTSTEEGTSTEPSEGSAPGTSTEPSEGSAPGSEPA | |
| | TSGSETPGTSESATPESGPGTSTEPSEGSAPGFPTIPLSRLFDNAMLRA | |
| | HRLHQLAFDTYQEFEEAYIPKEQKYSFLQNPQTSLCFSESIPTPSNRE | |
| | ETQQKSNLELLRISLLLIQSWLEPVQFLRSVFANSLVYGASDSNVYD | |
| | LLKDLEEGIQTLMGRLEDGSPRTGQIFKQTYSKFDTNSHNDDALLK | |
| | NYGLLYCFRKDMDKVETFLRIVQCRSVEGSCGFGGTSESATPESGP | |
| | GTSTEPSEGSAPGTSTEPSEGSAPGTSESATPESGPGTSTEPSEGSAPG | |
| | TSTEPSEGSAPGTSESATPESGPGTSTEPSEGSAPGTSTEPSEGSAPGT | |
| | STEPSEGSAPGSPAGSPTSTEEGTSTEPSEGSAPG | |
| AE912. | AEPAGSPTSTEEGTPGSGTASSSPGSSTPSGATGSPGASPGTSSTGSP | 5 |
| 2-hGH- | GSPAGSPTSTEEGTSESATPESGPGTSTEPSEGSAPGSPAGSPTSTEEG | |
| G- | TSTEPSEGSAPGTSTEPSEGSAPGTSESATPESGPGSEPATSGSETPGS | |
| AE288- | EPATSGSETPGSPAGSPTSTEEGTSESATPESGPGTSTEPSEGSAPGTS | |
| PATSG | TEPSEGSAPGSPAGSPTSTEEGTSTEPSEGSAPGTSTEPSEGSAPGTSE | |
| SETPG | SATPESGPGTSTEPSEGSAPGTSESATPESGPGSEPATSGSETPGTSTE | |
| TSESA | PSEGSAPGTSTEPSEGSAPGTSESATPESGPGTSESATPESGPGSPAGS | |
| TPESG | PTSTEEGTSESATPESGPGSEPATSGSETPGTSESATPESGPGTSTEPS | |
| PGTST | EGSAPGTSTEPSEGSAPGTSTEPSEGSAPGTSTEPSEGSAPGTSTEPSE | |
| EPSEG | GSAPGTSTEPSEGSAPGSPAGSPTSTEEGTSTEPSEGSAPGTSESATPE | |
| SAPG | SGPGSEPATSGSETPGTSESATPESGPGSEPATSGSETPGTSESATPES | |
| (″PAT | GPGTSTEPSEGSAPGTSESATPESGPGSPAGSPTSTEEGSPAGSPTSTE | |
| SGSET | EGSPAGSPTSTEEGTSESATPESGPGTSTEPSEGSAPGTSESATPESGP | |
| PGTS | GSEPATSGSETPGTSESATPESGPGSEPATSGSETPGTSESATPESGPG | |
| ESATP | TSTEPSEGSAPGSPAGSPTSTEEGTSESATPESGPGSEPATSGSETPGT | |
| ESGP | SESATPESGPGSPAGSPTSTEEGSPAGSPTSTEEGTSTEPSEGSAPGTS | |
| GTST | ESATPESGPGTSESATPESGPGTSESATPESGPGSEPATSGSETPGSEP | |
| EPSE | ATSGSETPGSPAGSPTSTEEGTSTEPSEGSAPGTSTEPSEGSAPGSEPA | |
| GSAP | TSGSETPGTSESATPESGPGTSTEPSEGSAPGFPTIPLSRLFDNAMLRA | |
| G″ | HRLHQLAFDTYQEFEEAYIPKEQKYSFLQNPQTSLCFSESIPTPSNRE | |
| dis- | ETQQKSNLELLRISLLLIQSWLEPVQFLRSVFANSLVYGASDSNVYD | |
| closed | LLKDLEEGIQTLMGRLEDGSPRTGQIFKQTYSKFDTNSHNDDALLK | |
| as (SEQ | NYGLLYCFRKDMDKVETFLRIVQCRSVEGSCGFGGTSESATPESGP | |
| ID NO: | GSEPATSGSETPGTSESATPESGPGSEPATSGSETPGTSESATPESGPG | |
| 6) | TSTEPSEGSAPGSPAGSPTSTEEGTSESATPESGPGSEPATSGSETPGT | |
| | SESATPESGPGSPAGSPTSTEEGSPAGSPTSTEEGTSTEPSEGSAPGTS | |
| | ESATPESGPGTSESATPESGPGTSESATPESGPGSEPATSGSETPGSEP | |
| | ATSGSETPGSPAGSPTSTEEGTSTEPSEGSAPGTSTEPSEGSAPGSEPA | |
| | TSGSETPGTSESATPESGPGTSTEPSEGSAPG | |

Further characterization of the exemplary hGH-XTEN fusion proteins provided in Table 1 can be found in the examples (e.g., Examples 27-35) of Schellenberger et al. WO10/144502A2 and U.S. Pat. No. 8,703,717, which are incorporated herein by reference in its entirety.

The GH of the subject compositions, together with their corresponding nucleic acid and amino acid sequences, are well known in the art and descriptions and sequences are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences may be a wild type polynucleotide sequence encoding a given GH (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species; or a polynucleotide encoding a variant of the wild type protein, such as a site directed mutant or an allelic variant. It is well within the ability of the skilled artisan to use a wild-type or consensus cDNA sequence or a codon-optimized variant of a GH to create fusion protein constructs contemplated by the invention using methods known in the art and/or in conjunction with the guidance and methods provided herein, and described more fully in the Examples of Schellenberger et al. WO10/144502A2 and U.S. Pat. No. 8,703,717, which are incorporated herein by reference in its entirety.

The GH for inclusion in the hGH-XTEN of the invention include any growth hormone or sequence variant of biologic, therapeutic, prophylactic, or diagnostic interest or function, or that is useful for mediating or preventing or ameliorating a disease, disorder or condition associated with growth, growth hormone deficiency or defect when administered to a subject. Of particular interest are hGH-XTEN fusion protein compositions for which an increase in a pharmacokinetic parameter, increased solubility, increased stability, or some other enhanced pharmaceutical or pharmacodynamic property compared to native GH is sought, or for which increasing the terminal half-life would improve efficacy, safety, or result in reduce dosing frequency and/or improve patient compliance. Thus, the hGH-XTEN fusion protein compositions are prepared with various objectives in mind, including improving the therapeutic efficacy of the bioactive GH by, for example, increasing the in vivo exposure or the length that the hGH-XTEN remains within the therapeutic window when administered to a subject, compared to a GH not linked to XTEN.

In one embodiment, the GH incorporated into the subject compositions can be a recombinant polypeptide with a sequence corresponding to a protein found in nature, such as human growth hormone. In one embodiment, the GH is human GH comprising the following amino acid sequence:

(SEQ ID NO: 2)
FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF.

In another embodiment, the GH is a sequence variant, fragment, homolog, or mimetic of a natural sequence that retain at least a portion of the biological activity of the native GH. In non-limiting examples, a GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the protein sequence of SEQ ID NO: 2. In one embodiment, the hGH-XTEN fusion protein comprises a single GH molecule linked to an XTEN. In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO: 2), and the first and the second XTEN are sequences that exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence selected to the C-terminus XTEN domain of SEQ ID NO: 1, the N-terminus XTEN domain of SEQ ID NO: 1, and any of the XTEN domains from Table 1.

### VI). Uses of the Compositions of the Present Invention

In one aspect, the invention provides a method for achieving a beneficial effect in a Growth hormone deficiency” or “GHD” in a human patient. “Growth hormone deficiency” or “GHD” as used herein refers to a disease, deficiency, disorder or condition in a human patient that would benefit from treatment with growth hormone. GHD includes disorders that are classified based on the source of the GH deficiency (e.g., pituitary GHD, hypothalamic GHD, functional GHD, and idiopathic GHD). Pituitary or “classic” GHD is the incapacity of the pituitary to produce growth hormone. “Hypothalamic GHD” is the failure of the hypothalamus to produce and/or transmit the neuroendocrine messaging hormone, growth hormone releasing hormone (GHRH), which directs a properly functioning pituitary to produce GH; “functional GHD” is the failure of other hormone and of metabolic functions related to the failure of the pituitary to produce, uptake, and/or utilize GH.

In one embodiment, the human patient having a GHD is an adult. GHD includes “adult growth hormone deficiency” or “AGHD”, which may be classified based on the stage of life the GH deficiency became manifest. For example, an adult may have AGHD that is a continuation of childhood onset GHD (including child-onset GHD and child-onset idiopathic GHD), which began in infancy or childhood. The causes of childhood-onset AGHD include, without limitation, developmental defects in or near the pituitary gland; genetic problems with the production of GH; Prader-Willi syndrome; Turner's syndrome; midline facial defects; and damage to the pituitary gland or the surrounding area due to tumors, infection, radiation treatment, or severe head injury. Adults who survived brain tumors as children may be at risk of developing GHD from the effects of surgery, cranial radiation or chemotherapy.

AGHD can develop in an adult, i.e., adult-onset GHD, (including adult-onset GHD and idiopathic adult onset-GHD) who was not diagnosed as being GH-deficient as a child. Adult-onset AGHD may be caused by damage or trauma to the pituitary gland. The damage is typically caused by a tumor (e.g., a tumor in and/or around the pituitary gland; or a tumor in the hypothalamus). Pituitary tumors can compress the gland or damage can occur when the tumor is removed via neurosurgery. The pituitary can also be damaged by infection, blood vessel disease, severe head injury, or cranial radiation or chemotherapy for treating tumors of the head and neck. AGHD may be caused by: trauma that occurred in an adult at their birth or soon after their birth; central nervous system infection; tumors of the hypothalamus or pituitary glands; infiltrative or granulomatous disease; cranial irradiation; surgery; or idiopathic causes. GHD in the elderly becomes manifest in decreased quality of life, fatigue, and alteration of body composition. Abnormalities in body composition, bone metabolism, and lipid profile in GH-deficient and hypopituitary adults are distinct from those that occur as the result of normal aging. AGHD includes congenital or acquired GH deficiency in adults, as well as any other adult indication for which GH can be utilized (including where endogenous growth hormone levels in a subject are not necessarily deficient).

Most processes involved in growth of the body are regulated by multiple peptides and hormones, and such peptides and hormones, as well as analogues thereof, have found utility in the treatment of growth hormone-related diseases, disorders and conditions. However, the use of commercially-available growth hormones, has met with less than optimal success in the management of subjects afflicted with such diseases, disorders and conditions. In particular, dose optimization and frequency of dosing is important for peptide and hormone biologics used in the treatment of growth hormone-related diseases and disorders. Safe and effective dosing requires considering and adjusting for a complicated set of factors—related to, for example, the drug's efficacy, safety, and complex metabolism. The fact that growth hormone has a short half-life (e.g., usually less than 4 hours when administered subcutaneously), necessitates frequent (e.g., daily) dosing in order to achieve clinical benefit, which results in difficulties in the management of such patients. Non-compliance with daily GH injections can lead to loss of treatment effects.

In some embodiments, the present invention relates to the enhancement of the safety and tolerability, and the ability to achieve IGF-I levels within a target range in adults with GHD after administration of the long-acting rhGH analogue, VRS-317, the sequence of which is shown in FIG. 1 (SEQ ID NO: 1). VRS-317 is an investigational rhGH analog, designed to maintain active drug levels for a longer period of time than currently available therapies. It is being developed for treatment of GHD in adults and children.

The first in human study of VRS-317 was a randomized, placebo controlled, single ascending dose study of 5 active dosing groups in 50 adult subjects with documented GHD. VRS-317 doses of 0.05, 0.10, 0.20, 0.40, or 0.80 mg/kg were given as a single subcutaneous (SC) dose. Each of the five dosing arms consisted of 8 subjects randomized to active drug and 2 subjects to placebo. Blood samples for PK and PD determinations were obtained at 21 time points over 30 days. The amplitude and duration of IGF-I exposure was directly proportional to the VRS-317 dose. IGF-I SDS was normalized for a mean of approximately three weeks for the 0.80 mg/kg group. Time to maximum concentration of IGF-I in this group was 5.1±2.0 days. Safety monitoring was carried out for 60 days post-dose.

Single doses of VRS-317 in adults with GHD were safe and well tolerated. There were no serious or unexpected adverse events (AEs). There was no lipoatrophy or nodule formation at the injection sites. There were no laboratory safety signals. All subjects completed the study.

Single VRS-317 doses safely increased the amplitude and duration of IGF-I in a dose dependent manner. After a single 0.80 mg/kg dose, mean IGF-I SDS was maintained in the therapeutic range (greater than −1.50 SDS) for a mean of three weeks. At 0.80 mg/kg, VRS-317 had a mean terminal elimination half-life (t_{1/2}) of 131 hours, 30-60 times longer than reported for daily rhGH in approved product package inserts. Prolonged IGF-I responses were achieved without meaningful overexposure to IGF-I. The pharmacokinetics and pharmacodynamics combined with the observed safety profile indicate the potential for safe and effective monthly dosing.

In a second study, an initial starting dose of 0.6 to 1.0 mg/kg was given to the participants in the study with subsequent 5 doses one monthly. The results from this study surprisingly indicated that dosing initiation and titration should be made on a fixed (mg) basis and do not need to be based upon-body weight adjusted (mg/kg) basis or the age of the patients.

Thus, in one embodiment, the invention provides a safe and effective method for the treatment of a growth hormone deficiency in a human patient comprising administering to the human patient a composition comprising a long-acting growth hormone. In another embodiment, the patient is an adult. In another embodiment, the patient is an adult with adult growth hormone deficiency (AGHD). In another embodiment, the long-acting growth hormone is an rhGH analogue. In another embodiment, the composition comprising the long-acting growth hormone is administered to a human patient as an initial dose and a plurality of subsequent doses. In another embodiment, the composition comprising the long-acting growth hormone is administered to a human patient as a plurality of doses for at least about 6 months or for at least about 12 months following an initial administration. In another embodiment, the long-acting growth hormone is dosed at between about 5 mg/administration and about 500 mg/administration (i.e., about 5 mg per administration and about 500 mg per administration). In another embodiment, the composition comprising the long-acting growth hormone is dosed once a month (30 days±2 days), twice a month (15 days±2 days), three times a month (10 days±2 days) or four times a month (7 days±2 days). In a preferred embodiment, the dose of the long-acting growth hormone administered per administration is not based on the body weight of the human patient. In one embodiment, the dose of the long-acting growth hormone administered per administration is based on the IGF-I standard deviation score (SDS) in a plasma or serum sample obtained from the patient following a previous administration of the composition comprising the long-acting growth hormone. In a preferred embodiment, the long-acting growth hormone is a human growth hormone-XTEN (hGH-XTEN) fusion protein. In one embodiment, the human growth hormone-XTEN (hGH-XTEN) fusion protein comprises one or more XTEN sequences on the N-terminal, C-terminal, or both terminal ends of the rhGH. In another embodiment, the hGH-XTEN fusion protein comprises a single GH molecule linked to a first and/or a second XTEN sequence, with an N- to C-terminus configuration of XTEN-GH, GH-XTEN, or XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO: 2), and a first and a second XTEN sequences exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence selected from the C-terminus XTEN domain of SEQ ID NO: 1, the N-terminus XTEN domain of SEQ ID NO: 1, or any of the XTEN domains from Table 1. In another embodiment, the hGH-XTEN fusion protein has at least 90% sequence identity to SEQ ID NO:1.

In other embodiments, the invention provides a method for the treatment of AGHD comprising administering to a human patient with AGHD a human growth hormone-XTEN (hGH-XTEN) fusion protein comprising an amino acid sequence having at least 90% sequence identity to SEQ ID NO:1 at a dose between about 5 mg/administration and about 500 mg/administration.

In some embodiments, the invention provides a method for the treatment of AGHD comprising administering to a human patient with AGHD a hGH-XTEN fusion protein at a dose between about 5 mg/administration and about 500 mg/administration. In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO: 2), and the first and the second XTEN are sequences that exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence selected from the C-terminus XTEN domain of SEQ ID NO: 1, the N-terminus XTEN domain of SEQ ID NO: 1, and any of the XTEN domains from Table 1.

In another embodiment, the invention provides a method for the treatment of AGHD comprising administering to a human patient with AGHD a hGH-XTEN fusion protein at a dose between about 5 mg/administration and about 500 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 5 mg/administration and about 500 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 5 mg/administration and about 500 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1.

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 10 mg/administration and about 400 mg/administration. In another embodiment, the invention provides a method for the treatment of AGHD comprising administering to a human patient with AGHD a hGH-XTEN fusion protein at a dose between about 10 mg/administration and about 400 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 10 mg/administration and about 400 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. In some embodiments, the invention provides a method for the treatment of AGHD comprising administering to a human patient with AGHD a hGH-XTEN fusion protein comprising the amino acid sequence of SEQ ID NO:1 at a dose between about 10 mg/administration and about 400 mg/administration.

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 400 mg/administration. In another embodiment, the invention provides a method for the treatment of AGHD comprising administering to a human patient with AGHD a hGH-XTEN fusion protein at a dose between about 20 mg/administration and about 400 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 400 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. In some embodiments, the invention provides a method for the treatment of AGHD comprising administering to a human patient with AGHD a hGH-XTEN fusion protein comprising the amino acid sequence of SEQ ID NO:1 at a dose between about 20 mg/administration and about 400 mg/administration.

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 300 mg/administration. In another embodiment, the invention provides a method for the treatment of AGHD comprising administering to a human patient with AGHD a hGH-XTEN fusion protein at a dose between about 20 mg/administration and about 300 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 300 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. In some embodiments, the invention provides a method for the treatment of AGHD comprising administering to a human patient with AGHD a hGH-XTEN fusion protein comprising the amino acid sequence of SEQ ID NO:1 at a dose between about 20 mg/administration and about 300 mg/administration.

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 250 mg/administration. In another embodiment, the invention provides a method for the treatment of AGHD comprising administering to a human patient with AGHD a hGH-XTEN fusion protein at a dose between about 20 mg/administration and about 250 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 250 mg/administration, wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. In some embodiments, the invention provides a method for the treatment of AGHD comprising administering to a human patient with AGHD a hGH-XTEN fusion protein comprising the amino acid sequence of SEQ ID NO:1 at a dose between about 20 mg/administration and about 250 mg/administration.

In another aspect, the dose of the hGH-XTEN fusion protein is administered to a human patient on a regular basis over a suitable time period, which can be finite or indefinite. In one embodiment, the hGH-XTEN dose is administered every week, every two weeks, every three weeks, or monthly. In other embodiments, the hGH-XTEN dose is administered once a month, twice a month, three times a month, or four times a month. In another embodiment, the hGH-XTEN dose is administered about every 7 days, about every 10 days, about every 14 days, about every 21 days, about every 28 days, or about every 30 days. In one embodiment, the hGH-XTEN dose is administered on a non-daily basis, or is a non-daily dose.

In some embodiments, the dosing frequency of the hGH-XTEN fusion protein is once a month (30 days±2 days), twice a month (15 days±2 days), three times a month (10 days±2 days) or four times a month (7 days±2 days). In some embodiments, the dosing frequency is twice a month (15 days±2 days).

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 5 mg/administration and about 500 mg/administration, wherein the dosing frequency is twice a month (15 days±2 days). In one embodiment, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 5 mg/administration and about 500 mg/administration, and as a plurality of subsequent doses between about 5 mg/administration and about 500 mg/administration. In a preferred embodiment, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 5 mg/administration and about 500 mg/administration, and as a plurality of subsequent doses between about 5 mg/administration and about 500 mg/administration, wherein the dosing frequency is twice a month (15 days±2 days).

In another embodiment, the invention provides a method for the treatment of AGHD comprising administering to a human patient with AGHD a hGH-XTEN fusion protein at a dose between about 5 mg/administration and about 500 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 5 mg/administration and about 500 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. These embodiments further comprise a plurality of subsequent doses following an initial dose, wherein the plurality of subsequent doses between about 5 mg/administration and about 500 mg/administration, and wherein the plurality of subsequent doses are administered at a dosing frequency of twice a month (15 days±2 days).

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 10 mg/administration and about 400 mg/administration, wherein the dosing frequency is twice a month (15 days+2 days). In some embodiments, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 10 mg/administration and about 400 mg/administration, and as a plurality of subsequent doses between about 10 mg/administration and about 400 mg/administration, wherein the dosing frequency is twice a month (15 days±2 days).

In another embodiment, the invention provides a method for the treatment of AGHD comprising administering to a human patient with AGHD a hGH-XTEN fusion protein at a dose between about 10 mg/administration and about 400 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 10 mg/administration and about 400 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. These embodiments further comprise a plurality of subsequent doses following an initial dose, wherein the plurality of subsequent doses is between about 10 mg/administration and about 400 mg/administration, wherein the dosing frequency is twice a month (15 days+2 days).

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 400 mg/administration, wherein the dosing frequency is twice a month (15 days±2 days). In some embodiments, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 20 mg/administration and about 400 mg/administration, and as a plurality of subsequent doses between about 20 mg/administration and about 400 mg/administration, wherein the dosing frequency is twice a month (15 days±2 days).

In another embodiment, the invention provides a method for the treatment of AGHD comprising administering to a human patient with AGHD a hGH-XTEN fusion protein at a dose between about 20 mg/administration and about 400 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 400 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. These embodiments further comprise a plurality of subsequent doses following an initial dose, wherein the plurality of subsequent doses are between about 20 mg/administration and about 400 mg/administration, and wherein the dosing frequency is twice a month (15 days±2 days).

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 300 mg/administration, wherein the dosing frequency is twice a month (15 days±2 days). In some embodiments, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 20 mg/administration and about 300 mg/administration, and as a plurality of subsequent doses between about 20 mg/administration and about 300 mg/administration, wherein the dosing frequency is twice a month (15 days±2 days).

In another embodiment, the invention provides a method for the treatment of AGHD comprising administering to a human patient with AGHD a hGH-XTEN fusion protein at a dose between about 20 mg/administration and about 300 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 300 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. These embodiments further comprise a plurality of subsequent doses following an initial dose, wherein the plurality of subsequent doses are between about 20 mg/administration and about 300 mg/administration, and wherein the dosing frequency is twice a month (15 days±2 days).

In some embodiments, the invention provides a method for the treatment of AGHD comprising administering to a human patient with AGHD a hGH-XTEN fusion protein comprising an amino acid sequence having at least 90% sequence identity to SEQ ID NO:1 at a dose between about 20 mg/administration and about 250 mg/administration, wherein the dosing frequency is twice a month (15 days±2 days). In some embodiments, the invention provides a method for the treatment of AGHD comprising administering to a human patient with AGHD a hGH-XTEN fusion protein comprising an amino acid sequence having at least 90% sequence identity to SEQ ID NO:1 at an initial dose between about 20 mg/administration and about 250 mg/administration, and as a plurality of subsequent doses between about 20 mg/administration and about 250 mg/administration, wherein the dosing frequency is twice a month (15 days±2 days).

In another embodiment, the invention provides a method for the treatment of AGHD comprising administering to a human patient with AGHD a hGH-XTEN fusion protein at a dose between about 20 mg/administration and about 250 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the invention provides a method for the treatment of AGHD comprising administering to a human patient with AGHD a hGH-XTEN fusion protein at a dose between about 20 mg/administration and about 250 mg/administration, wherein the dosing frequency is twice a month (15 days±2 days), and wherein the hGH-XTEN fusion protein comprises a single GH molecule linked to a first and a second XTEN, with an N- to C-terminus configuration of XTEN-GH-XTEN, in which the GH is a sequence that exhibits at least about 95% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, the N-terminus XTEN domain of SEQ ID NO: 1, and any of the XTEN domains from Table 1. These embodiments further comprise a plurality of subsequent doses between about 20 mg/administration and about 250 mg/administration, wherein the dosing frequency is twice a month (15 days±2 days).

The methods of the present invention are advantageous with respect to IGF-I levels in the human patient following treatment with hGH-XTEN fusion protein. A high level of blood IGF-I is undesirable since high IGF-I is believed to be a risk factor for cancer (Svensson et al. J Clin Endocrin Metab. epub Sep. 26, 2012 as doi:10.1210/jc.2012-2329). IGF-I generation in humans is largely the result of GH signaling and IGF-I is an important mediator for anabolic actions observed during GH therapy (Le Roith et al. (2001). Endocr Rev 22, 53-74). Accordingly, IGF-I is an important pharmacodynamic marker for hGH-XTEN fusion protein bioactivity. In practice, IGF-I responses to GH (e.g., daily rhGH therapy) are interpreted in terms of age- and gender-specific normative data (Vance et al. (1999). N Engl J Med 341, 1206-16; Molitch et al. (2011). J Clin Endocrinol Metab 96, 1587-609). The interpretation is most readily done with the use of IGF-I standard deviation scores (IGF-I SDS). Further, adults with GH deficiency, as with healthy adults, have a range of baseline IGF-I values. Accordingly, IGF-I SDS, corrected for baseline at time 0, can be used to examine potential hGH-XTEN fusion protein dose effects on IGF-I responses.

In one aspect, the present invention provides methods of treatment of GHD in which the human patient maintains an IGF-I response (e.g., as measured by mean IGF-I SDS) in a normal range after administration of the hGH-XTEN fusion protein. For an IGF-I SDS, a normal range is generally between about −1.5 and about 1.5 but can also be between about −2.0 and about 2.0.

It should be noted that where reference is made to an IGF-I SDS between about a first value (e.g., −2.0) and about a second value (e.g., 2.0), the “first value” may include the first value and the “second value” may include the second value.

In some embodiments, the human patient has a serum IGF-I standard deviation score (SDS) between about −2.0 and about 2.0 following administration of a composition comprising a long-acting growth hormone, preferably a composition comprising hGH-XTEN. In one other embodiment, the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In an additional embodiment, the human patient exhibits said serum IGF-I SDS following administration of a dose of the composition comprising the long-acting growth hormone, wherein the dosing frequency is once a month (30 days±2 days), twice a month (15 days±2 days), three times a month (10 days±2 days) or four times a month (7 days±2 days). In some embodiments, the dosing frequency is twice a month (15 days±2 days). In one embodiment, the administration of the hGH-XTEN results in a normalization of IGF-I SDS in the human patient for at least about 7 days, at least about 10 days, at least about 14 days, at least about 16 days, or at least about 21 days.

In some embodiments, the hGH-XTEN dose is effective to maintain the patient's serum IGF-I standard deviation score (SDS) between about −2.0 and about 2.0 for at least 7 days after administration of the hGH-XTEN at a dose between about 5 mg/administration and about 500 mg/administration. In other embodiments, the hGH-XTEN dose is (i) between about 10 mg/administration and about 400 mg/administration; (ii) between about 20 mg/administration and about 400 mg/administration; (iii) between about 20 mg/administration and about 300 mg/administration; or (iv) between about 20 mg/administration and about 250 mg/administration. In another embodiment, the hGH-XTEN dose is effective to maintain the patient's serum IGF-I SDS between about −2.0 and about 2.0 for at least 14 days after administration of the dose. In one embodiment, the hGH-XTEN dose is administered subcutaneously.

In one additional aspect, the present invention provides methods for improving dosing of hGH therapy in a human patient. In one embodiment, the hGH therapy comprises administering a composition comprising a long-acting growth hormone, preferably a composition comprising hGH-XTEN. In another aspect, the present invention provides methods of determining a subsequent dose of an hGH-XTEN fusion protein administered over a subsequent dosage period when treating a human patient with AGHD with the hGH-XTEN fusion protein. The “dosage period” means the time between the administration of a dose (e.g., initial dose) and the next successive administration of a dose (e.g., subsequent dose). The dosage period may change with one or more further successive dose or doses, or may remain constant.

In some embodiments, the invention provides a method for the treatment of AGHD comprising administering to a human patient with growth hormone deficiency a hGH-XTEN at a starting dose followed by subsequent doses that are titrated for each individual patient until each patient achieves their optimal maintenance dose. In one embodiment, the human patient is an adult having AGHD.

In some embodiments, the foregoing methods comprise the step of monitoring the IGF-I standard deviation score (SDS) in a plasma or serum sample obtained from the patient during an initial dosage period of administration of an initial dose of a long-acting growth hormone, preferably hGH-XTEN. In one embodiment, the hGH-XTEN fusion protein comprising an amino acid sequence having at least about 90% sequence identity to SEQ ID NO:1. In another embodiment, the method further comprises the step of determining a subsequent dose of hGH-XTEN fusion protein (or another long-acting growth hormone) administered over a subsequent dosage period based on the IGF-I SDS observed during the initial dosage period. In one additional embodiment, the method further comprises administering the subsequent dose over a subsequent dosage period. In one other embodiment, the subsequent dose improves the efficacy of the treatment during the subsequent dosage period. In another embodiment, the subsequent dose is higher, lower, or equivalent to the initial dose. The initial dose or subsequent dose may be any of the doses described herein. In one additional embodiment, the subsequent dosage period is longer, shorter, or equivalent to the initial dosage period.

In some embodiments, a starting dose for a long-acting growth hormone is selected regardless of weight, age and gender of the adult patient. In some embodiments, the long-acting growth hormone comprises hGH-XTEN and a starting dose for the hGH-XTEN is selected regardless of weight, age and gender of the adult patient. In some embodiments, a starting dose for the hGH-XTEN is selected from (i) 20 mg for all subjects other than women receiving oral estrogen or (ii) 40 mg for women receiving oral estrogen. The hGH-XTEN dose is titrated until a maintenance dose is achieved. In some embodiments, the dosing frequency is once a month (30 days±2 days), twice a month (15 days±2 days), three times a month (10 days±2 days) or four times a month (7 days±2 days). In some embodiments, the dosing frequency is twice a month (15 days±2 days).

In some embodiments, the doses are titrated to each patient's individual IGF-I responses based on the IGF-I level 7 days post-dose until a maintenance dose is achieved. In some embodiments, the maintenance dose is an IGF-I value between −2.0 and 2.0 SDS for two consecutive 7 day post-dose time points (Day 8, peak level). In some embodiments, the maintenance dose is an IGF-I value between −1.0 and 2.0 SDS for two consecutive 7 day post-dose time points (Day 8, peak level). In some embodiments, the maintenance dose is an IGF-I value between −1.5 and 2.0 SDS for two consecutive 7 day post-dose time points (Day 8, peak level). In some embodiments, the maintenance dose is an IGF-I value between 0 and 2.0 SDS for two consecutive 7 day post-dose time points (Day 8, peak level).

In some embodiments, the doses are titrated per the Dose Titration Plan described in Table 2. In some embodiments, the doses may be modified based on the medical professional's (e.g. patient's doctors) clinical judgment. In some embodiments, after the patients achieve maintenance dosing (e.g., two consecutive IGF-I at Day 8 within target range under the same dose), doses may be modified based on the medical professional (e.g. patient's doctor or Medical Monitor clinical judgment), for example, with the goal to maintain IGF-I SDS at Day 8 within target range (0 to 2.0).

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at a dose between about 5 mg/administration and about 500 mg/administration, wherein the dosing frequency is twice a month (15 days±2 days), and further wherein said amount is effective to maintain the subject's serum IGF-I SDS between about −1.5 and about 1.5 for (i) at least 7 days; (ii) at least about 10 days; or (iii) at least about 20 days after administration of the single dose of the fusion protein. In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 5 mg/administration and about 500 mg/administration, and as a plurality of subsequent doses between about 5 mg/administration and about 500 mg/administration, wherein the dosing frequency is twice a month (15 days+2 days) and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at a dose between about 10 mg/administration and about 400 mg/administration, wherein the dosing frequency is twice a month (15 days±2 days), and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein. In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 10 mg/administration and about 400 mg/administration, and as a plurality of subsequent doses between about 10 mg/administration and about 400 mg/administration, wherein the dosing frequency is twice a month (15 days±2 days) and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at a dose between about 20 mg/administration and about 400 mg/administration, wherein the dosing frequency is twice a month (15 days±2 days), and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein. In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 20 mg/administration and about 400 mg/administration, and as a plurality of subsequent doses between about 20 mg/administration and about 400 mg/administration, wherein the dosing frequency is twice a month (15 days±2 days) and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at a dose between about 20 mg/administration and about 300 mg/administration, wherein the dosing frequency is twice a month (15 days+2 days), and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein. In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 20 mg/administration and about 300 mg/administration, and as a plurality of subsequent doses between about 20 mg/administration and about 300 mg/administration, wherein the dosing frequency is twice a month (15 days±2 days) and further wherein said amount is effective to maintain the subject's serum IGF-I SDS between about −1.5 and about 1.5 for (i) at least 7 days; (ii) at least about 10 days; or (iii) at least about 20 days after administration of the single dose of the fusion protein.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at a dose between about 20 mg/administration and about 250 mg/administration, wherein the dosing frequency is twice a month (15 days±2 days), and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein. In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 20 mg/administration and about 250 mg/administration, and as a plurality of subsequent doses between about 20 mg/administration and about 250 mg/administration, wherein the dosing frequency is twice a month (15 days±2 days) and further wherein said amount is effective to maintain the subject's serum IGF-I SDS between about −1.5 and about 1.5 for (i) at least 7 days; (ii) at least about 10 days; or (iii) at least about 14 days after administration of the single dose of the fusion protein.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose between about 20 mg if the patient is not a woman receiving estrogen or 40 mg if the patient is a women receiving estrogen, and a plurality of subsequent doses, wherein said subsequent doses are increased or decreased to achieve a maintenance dose, wherein said maintenance dose is achieved when the patient's serum IGF-I SDS is between about −2.0 and about 2.0 for two consecutive 7 days after administration, and wherein the dosing frequency is twice a month (15 days±2 days). In some embodiments, after the maintenance dose is achieved, the maintenance dose is effective to maintain the patient's serum IGF-I SDS between about −2.0 and about 2.0 for (i) at least 7 days; (ii) at least about 10 days; or (iii) at least about 20 days after administration.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose of about 20 mg if the patient is not a woman receiving estrogen or 40 mg if the patient is a women receiving estrogen, and a plurality of subsequent doses, wherein said subsequent doses are increased or decreased to achieve a maintenance dose, wherein said maintenance dose is achieved when the patient's serum IGF-I SDS is between about −1.5 and about 2.0 for two consecutive 7 days after administration, and wherein the dosing frequency is twice a month (15 days±2 days). In some embodiments, after the maintenance dose is achieved, the maintenance dose is effective to maintain the patient's serum IGF-I SDS between about −1.5 and about 2.0 for (i) at least 7 days; (ii) at least about 10 days; or (iii) at least about 20 days after administration.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose of about 20 mg if the patient is not a woman receiving estrogen or 40 mg if the patient is a women receiving estrogen, and a plurality of subsequent doses, wherein said subsequent doses are increased or decreased to achieve a maintenance dose, wherein said maintenance dose is achieved when the patient's serum IGF-I SDS is between about 0 and about 2.0 for two consecutive 7 days after administration, and wherein the dosing frequency is twice a month (15 days±2 days). In some embodiments, after the maintenance dose is achieved, the maintenance dose is effective to maintain the patient's serum IGF-I SDS between about −1.5 and about 2.0 for (i) at least 7 days; (ii) at least about 10 days; or (iii) at least about 20 days after administration.

In some embodiments, the invention provides a method for the treatment of AGHD comprising administering to a human patient with AGHD a pharmaceutical composition comprising an effective amount of hGH-XTEN fusion protein having the amino acid sequence set forth in SEQ ID NO:1 at an initial dose of about 20 mg if the patient is not a woman receiving estrogen or 40 mg if the patient is a women receiving estrogen, and a plurality of subsequent doses, wherein said subsequent doses are increased or decreased to achieve a maintenance dose, wherein said maintenance dose is achieved when the patient's serum IGF-I SDS is between about 1 and about 2.0 for two consecutive 7 days after administration, and wherein the dosing frequency is twice a month (15 days±2 days). In some embodiments, after the maintenance dose is achieved, the maintenance dose is effective to maintain the patient's serum IGF-I SDS between about 0 and about 2.0 for (i) at least 7 days; (ii) at least about 10 days; or (iii) at least about 20 days after administration.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 30 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 35 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is a woman receiving estrogen a pharmaceutical composition comprising an effective amount of hGH-XTEN fusion protein having an amino acid sequence having at least about 90% sequence identity to SEQ ID NO:1 at a dose between about 40 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is a woman receiving estrogen a pharmaceutical composition comprising an effective amount of hGH-XTEN fusion protein having an amino acid sequence having at least about 90% sequence identity to SEQ ID NO:1 at a dose between about 45 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is a woman receiving estrogen a pharmaceutical composition comprising an effective amount of hGH-XTEN fusion protein having an amino acid sequence having at least about 90% sequence identity to SEQ ID NO:1 at a dose between about 30 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 5 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 200 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 150 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 100 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 90 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 80 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 60 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 50 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD who is not a woman receiving estrogen a pharmaceutical composition comprising an effective amount of hGH-XTEN fusion protein having an amino acid sequence having at least about 90% sequence identity to SEQ ID NO:1 at a dose between about 10 mg/administration and about 40 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the starting dose and titration of a long-acting growth hormone can be based on the patient baseline IGF-1 levels and/or duration of disease. In some embodiments, the doses may be modified based on the medical professional's (e.g. patient's doctors) clinical judgment. In a preferred embodiment, the long-acting growth hormone comprises hGH-XTEN.

In some embodiments, the invention provides a method for the treatment of AGHD comprising administering to a human patient with AGHD having a baseline IGF-I SDS of less or equal than −1 a pharmaceutical composition comprising an effective amount of hGH-XTEN fusion protein having an amino acid sequence having at least about 90% sequence identity to SEQ ID NO:1 at a dose between about 10 mg/administration and about 250 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD having a baseline IGF-I SDS of less or equal than −1 a pharmaceutical composition comprising an effective amount of hGH-XTEN fusion protein having an amino acid sequence having at least about 90% sequence identity to SEQ ID NO:1 at a dose between about 10 mg/administration and about 150 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD having a baseline IGF-I SDS of less or equal than −1 a pharmaceutical composition comprising an effective amount of hGH-XTEN fusion protein having an amino acid sequence having at least about 90% sequence identity to SEQ ID NO:1 at a dose between about 10 mg/administration and about 80 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD having a baseline IGF-I SDS of less or equal than −1 a pharmaceutical composition comprising an effective amount of hGH-XTEN fusion protein having an amino acid sequence having at least about 90% sequence identity to SEQ ID NO:1 at a dose between about 20 mg/administration and about 80 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient is more than 4 years, more than 5 years, more than 10 years, more than 15 years or more than 17 years.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 20 mg/administration and about 60 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 20 mg/administration and about 50 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 20 mg/administration and about 40 mg/administration, wherein the dosing frequency is twice a month (15 days±2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 20 mg/administration and about 30 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 10 mg/administration and about 40 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In some embodiments, the invention provides a method for the treatment of AGHD comprising administering to a human patient with AGHD having a baseline IGF-I SDS of higher than −1 a pharmaceutical composition comprising an effective amount of hGH-XTEN fusion protein having an amino acid sequence having at least about 90% sequence identity to SEQ ID NO:1 at a dose between about 10 mg/administration and about 30 mg/administration, wherein the dosing frequency is twice a month (15 days+2 days). In some embodiments, the dose is effective to maintain the patient's serum IGF-I SDS between about 0 and about 2.0 for (i) at least 7 days; (ii) at least about 10 days; or (iii) at least about 20 days after administration. In some embodiments, the time since diagnosis for the AGHD patient 4 years or less.

In a further embodiment of the method, the effective amount of the hGH-XTEN fusion protein administered to a human patient is at least about 5 mg, about 10 mg, about 15 mg, about 20 mg, about 24 mg, about 28 mg, about 30 mg, about 32 mg, about 36 mg, about 40 mg, about 44 mg, about 48 mg, about 50 mg, about 52 mg, about 56 mg, about 60 mg, about 64 mg, about 68 mg, about 70 mg, about 72 mg, about 76 mg, about 80 mg, about 82 mg, about 88 mg, about 90 mg, about 92 mg, about 96 mg, about 100 mg, about 104 mg, about 108 mg, about 110 mg, about 112 mg, about 116 mg, about 120 mg, about 124 mg, about 128 mg, about 130 mg, about 132 mg, about 136 mg, about 140 mg, about 144 mg, about 148 mg, about 150 mg, about 152 mg, about 156 mg, about 160 mg, about 164 mg, about 168 mg, about 170 mg, about 172 mg, about 176 mg, about 180 mg, about 184 mg, about 188 mg, about 190 mg, about 192 mg, about 196 mg, about 200 mg, about 204 mg, about 208 mg, about 210 mg, about 212 mg, about 216 mg, about 220 mg, about 224 mg, about 228 mg, about 230 mg, about 232 mg, about 236 mg, about 240 mg, about 244, about 248 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg.

The methods of the present invention provides a particular advantage in that that the administration of a long-acting growth hormone (e.g., hGH-XTEN fusion protein) provides an observable and prolonged IGF-I response in the human patient (e.g., as measured by IGF-I SDS) that is not accompanied by, or at the expense of, over-exposure to high levels of IGF-I, which is undesirable. In other words, the IGF-I response is maintained at an elevated level that is still considered acceptable by current standards, e.g., as indicated by an IGF-I SDS of 1.5 or less, or an IGF-I SDS of 2.0 or less.

In one embodiment, the invention provides a method for achieving a beneficial effect in a human patient with growth hormone deficiency, comprising the step of administering to the subject a therapeutically-effective amount of a long-acting growth hormone (e.g., hGH-XTEN fusion protein), wherein said administration results in the improvement of one or more biochemical or physiological parameters or clinical endpoints associated with AGHD. The effective amount produces a beneficial effect in helping to treat (e.g., cure or reduce the severity) the deleterious effects of AGHD.

The methods of the invention include the administration to a human patient of successive or consecutive doses of a therapeutically effective amount of a long-acting growth hormone (e.g., hGH-XTEN fusion protein) for a period of time sufficient to achieve and/or maintain the desired parameter or clinical effect, and such consecutive doses of a therapeutically effective amount establishes the therapeutically effective dose regimen for the hGH-XTEN; i.e., the schedule for consecutively administered doses of the fusion protein composition, wherein the doses are given in therapeutically effective amounts to result in a sustained beneficial effect on any clinical sign or symptom, aspect, measured parameter or characteristic of a metabolic disease state or condition, including, but not limited to, those described herein. In one embodiment of the method, the parameters include but are not limited to IGF-I concentration, ratio of IGF-I/IGFBP-3, IGFBP3 concentration, change in weight, lean body mass, change in body mass index, total body fat (adipose fat/tissue), trunk fat, response to insulin challenge, rate of division of chondrocytes, chondrocyte numbers, bone density, bone age, bone growth, bone turnover, increase in epiphyseal plate width, reduction in cholesterol, reduction in triglycerides, and reduction in LDL. In one additional embodiment, the human patient is an adult.

In one embodiment, the pharmaceutical composition is administered at a therapeutically effective dose. In another embodiment, the pharmaceutical composition is administered using multiple consecutive doses using a therapeutically effective dose regimen (as defined herein) for the length of the dosing period.

In another embodiment, the invention provides a method of treating AGHD in a human subject, comprising administering to the subject with AGHD a pharmaceutical composition comprising an effective amount of hGH-XTEN fusion protein having the amino acid sequence set forth in FIG. 1 (SEQ ID NO:1) at a dose between about 5 mg/administration and about 500 mg/administration, wherein said dose is effective to maintain a plasma concentration of said fusion protein at more than about 10 ng/mL for a period of at least 10 days after administration of the fusion protein. In another embodiment, the invention provides a method of treating AGHD in a human subject, comprising administering to the subject with AGHD a pharmaceutical composition comprising an effective amount of hGH-XTEN fusion protein having the amino acid sequence set forth in FIG. 1 (SEQ ID NO:1) at a dose between about 10 mg/administration and about 400 mg/administration, wherein said dose is effective to maintain a plasma concentration of said fusion protein at more than about 10 ng/mL for a period of at least 10 days after administration of the fusion protein. In another embodiment, the invention provides a method of treating AGHD in a human subject, comprising administering to the subject with AGHD a pharmaceutical composition comprising an effective amount of hGH-XTEN fusion protein having the amino acid sequence set forth in FIG. 1 (SEQ ID NO:1) at a dose between about 20 mg/administration and about 400 mg/administration, wherein said dose is effective to maintain a plasma concentration of said fusion protein at more than about 10 ng/mL for a period of at least 10 days after administration of the fusion protein. In another embodiment, the invention provides a method of treating AGHD in a human subject, comprising administering to the subject with AGHD a pharmaceutical composition comprising an effective amount of hGH-XTEN fusion protein having the amino acid sequence set forth in FIG. 1 (SEQ ID NO:1) at a dose between about 20 mg/administration and about 300 mg/administration, wherein said dose is effective to maintain a plasma concentration of said fusion protein at more than about 10 ng/mL for a period of at least 10 days after administration of the fusion protein. In another embodiment, the invention provides a method of treating AGHD in a human subject, comprising administering to the subject with AGHD a pharmaceutical composition comprising an effective amount of hGH-XTEN fusion protein having the amino acid sequence set forth in FIG. 1 (SEQ ID NO:1) at a dose between about 20 mg/administration and about 250 mg/administration, wherein said dose is effective to maintain a plasma concentration of said fusion protein at more than about 10 ng/mL for a period of at least 10 days after administration of the fusion protein. In some embodiments, the hGH-XTEN fusion protein dose is effective to maintain a plasma concentration of said fusion protein at more than about 10 ng/mL for a period of at least 14 days after administration of the fusion protein.

In one embodiment, the invention provides a method of treating AGHD in a human subject comprising administering to the subject with AGHD a pharmaceutical composition comprising an effective amount of hGH-XTEN fusion protein having the amino acid sequence set forth in FIG. 1 (SEQ ID NO:1) wherein said amount is between about 5 mg/administration to 500 mg/administration and is effective in increasing the subject's plasma IGF-I SD score by at least 0.5 above the subject's baseline IGF-I SD score without causing a clinically significant level of side-effects selected from the group consisting of headache, arthralgia, myalgia, edema, nausea, and muscle fatigue after administration of the single dose of the fusion protein. As used herein, “clinically significant level of side-effects” means that the side-effects are not unexpected or are not serious adverse events. Side-effects that are mild and transient, even if one of headache, arthralgia, myalgia, edema, nausea, and muscle fatigue or those otherwise known to be associated with the administration of growth hormone, would not be considered a clinically significant level. In another embodiment of the method of treating AGHD, the amount administered is between about 10 mg/administration to 400 mg/administration. In another embodiment of the method of treating AGHD, the amount administered is between 20 mg/administration to 400 mg/administration. In another embodiment of the method of treating AGHD, the amount administered is between about 20 mg/administration to 300 mg/administration. In another embodiment of the method of treating GHD, the amount administered is between about 20 mg/administration to 250 mg/administration. In another embodiment of the method of treating AGHD, the dose is administered subcutaneously. In another embodiment of the method of treating AGHD, the pharmaceutical composition comprising the hGH-XTEN fusion protein is administered using two or more consecutive doses.

The invention provides methods to establish a dose regimen for the hGH-XTEN pharmaceutical compositions of the invention for human patients. The methods include administration of consecutive doses of a therapeutically effective amount of a long-acting growth hormone composition (e.g., hGH-XTEN fusion protein composition) using variable periods of time between doses to determine that interval of dosing sufficient to achieve and/or maintain the desired parameter, blood level or clinical effect; such consecutive doses of a therapeutically effective amount at the effective interval establishes the therapeutically effective dose regimen for the long-acting growth hormone composition (e.g., hGH-XTEN fusion protein composition) for a AGHD condition. Thus, in one aspect, the invention provides an hGH-XTEN composition for use in a treatment regimen that is therapeutically effective for AGHD.

In one other aspect, the treatment regimen results in a clinically significant reduction in the patient in at least one parameter related to the AGHD evaluation after administration of a long-acting growth hormone (e.g., hGH-XTEN fusion protein) dose. In one embodiment, the treatment regimen results in a reduction in the patient of at least one parameter selected from serum cholesterol, serum triglycerides, and serum low density lipoprotein (LDL) after administration of the long-acting growth hormone (e.g., hGH-XTEN fusion protein) dose. In another embodiment, the treatment regimen comprises administration of a hGH-XTEN dose is once a month (30 days±2 days), twice a month (15 days±2 days), three times a month (10 days±2 days) or four times a month (7 days+2 days).

In some embodiments, the methods of the present invention related to improved therapeutic regimens for AGHD therapy comprise improving lipid metabolism parameters in a subject in need, e.g., a human patient with AGHD. In one embodiment, the method of improving lipid parameters in a subject in need comprises administering an at least two therapeutically effective doses of a long-acting growth hormone (e.g., hGH-XTEN fusion protein), wherein the administration of said doses is once a month (30 days±2 days), twice a month (15 days±2 days), three times a month (10 days±2 days) or four times a month (7 days±2 days), and wherein the doses provide an improvement in lipid parameters in said subject. In one embodiment, the improvement in lipid parameters is an improvement selected from the group consisting of lower triglyceride levels, lower cholesterol, and lower LDL levels. In some embodiments, the dosing frequency is twice a month (15 days±2 days).

In some embodiments of the regimen, the human patient achieves an improvement after two or more doses in at least one parameter selected from bone density, bone growth, and increase in epiphyseal plate width. In some embodiments, the foregoing improvement(s) is at least about 10%, or at least about 20%, or at least about 30%, or at least about 40%, or at least about 50%, or at least about 60%, or at least about 70%, or at least about 80%, or at least about 90%. In another embodiment, the foregoing % improvement(s) is similar to, or not inferior to, an improvement achieved by a non-long-acting hGH. In a preferred embodiment, the foregoing % improvement(s) is similar to, or not inferior to, an improvement achieved by an hGH not linked to XTEN and administered daily using daily dosage equivalent amounts of hGH.

In another aspect, the invention provides an hGH-XTEN fusion protein for use in a treatment regimen for AGHD, which regimen comprises administering a hGH-XTEN fusion protein to a human patient.

In general, a hGH-XTEN dose is a dose administered within a short period of time. In another embodiment, the hGH-XTEN dose is administered within about 1 to about 30 minutes, about 1 to about 20 minutes, about 1 to about 15 minutes, about 1 to about 10 minutes, or about 1 to about 5 minutes. In one embodiment, the hGH-XTEN dose is administered within about 1 to about 5 minutes. In one other embodiment, the hGH-XTEN dose is a subcutaneous dose.

In yet another embodiment, the hGH-XTEN fusion protein comprises an amino acid sequence shown as set forth in FIG. 1 (SEQ ID NO:1). In other embodiments, the administration is subcutaneous administration.

In another aspect, the present invention provides methods of normalizing serum IGF-I levels in a subject in need thereof. In one embodiment, the method comprises administering a long-acting growth hormone (e.g., hGH-XTEN fusion protein) to a human patient at a dose that is effective in increasing the patient's IGF-I SDS by at least 0.5 or at least 1.0 above the subject's baseline IGF-I SDS. In another embodiment, the increase in IGF-I SDS is achieved in the absence of a clinically significant level of side-effects selected from the group consisting of headache, arthralgia, myalgia, edema, nausea, and muscle fatigue after administration of the dose. In one additional embodiment, the dose is (i) a therapeutically effective dose as described herein; and/or (ii) is administered subcutaneously.

In some embodiments, the present invention provides an hGH-XTEN fusion protein for use as a medicament, or for the treatment of AGHD. In some embodiments, the present invention provides the use of an hGH-XTEN fusion protein for the manufacture of a medicament for treating GHD in a human patient with AGHD. In some embodiments, the present invention provides the use of the fusion protein having the sequence set forth in FIG. 1 (SEQ ID NO:1) in the manufacture of a medicament for the treatment of GHD. In some embodiments, the hGH-XTEN fusion protein is provided in a therapeutically effective dose suitable for administration. In some embodiments, the therapeutically effective dose of hGH-XTEN fusion protein is selected from the group consisting of: about 5 mg, about 6 mg, about 8 mg, about 10 mg, about 12 mg, about 14 mg, about 16 mg, about 18 mg, about 20 mg, about 24 mg, about 28 mg, about 30 mg, about 32 mg, about 36 mg, about 40 mg, about 44 mg, about 48 mg, about 50 mg, about 52 mg, about 56 mg, about 60 mg, about 64 mg, about 68 mg, about 70 mg, about 72 mg, about 76 mg, about 80 mg, about 82 mg, about 88 mg, about 90 mg, about 92 mg, about 96 mg, about 100 mg, about 104 mg, about 108 mg, about 110 mg, about 112 mg, about 116 mg, about 120 mg, about 124 mg, about 128 mg, about 130 mg, about 132 mg, about 136 mg, about 140 mg, about 144 mg, about 148 mg, about 150 mg, about 152 mg, about 156 mg, about 160 mg, about 164 mg, about 168 mg, about 170 mg, about 172 mg, about 176 mg, about 180 mg, about 184 mg, about 188 mg, about 190 mg, about 192 mg, about 196 mg, about 200 mg, about 204 mg, about 208 mg, about 210 mg, about 212 mg, about 216 mg, about 220 mg, about 224 mg, about 228 mg, about 230 mg, about 232 mg, about 236 mg, about 240 mg, about 244, about 248 mg, about 250 mg. In another embodiment, the therapeutically effective hGH-XTEN fusion protein dose is administered subcutaneously. In some embodiments, the human patient has a serum IGF-I standard deviation (SD) score of greater than about −2.0, greater than about −1.5, greater than about −1.0, greater than about −0.5, greater than about 0, greater than about 0.5, greater than about 1.0, greater than about 1.5, greater than about 1.6, greater than about 1.7, greater than about 1.8, or greater than about 1.9 following administration of the hGH-XTEN fusion protein. In one embodiment, the hGH-XTEN fusion protein comprises an amino acid sequence shown as set forth in FIG. 1 (SEQ ID NO:1).

In another aspect, the present invention provides hGH-XTEN fusion protein-based therapeutic agents for treating diseases or conditions related to AGHD. For the prevention, treatment or reduction in the severity of a given disease or condition, the appropriate dosage of a therapeutic agent of the invention will depend on the type of disease or condition to be treated, as defined above, the severity and course of the disease or condition, whether the agent is administered for therapeutic purposes, previous therapy, the patient's clinical history and response to the agent, and the discretion of the attending physician.

In another aspect, the present invention provides a method for the delaying or slowing down of the progression of a disease or condition related to AGHD. In one embodiment, the method comprises administering to subject diagnosed with the disease, condition, or disorder, an effective amount of an hGH-XTEN fusion protein. In another aspect, the invention provides a method for treating or ameliorating indicia of a disease or condition related to AGHD. In one embodiment, the method comprises administering an effective amount of an hGH-XTEN fusion protein to a subject at risk of the disease or condition, wherein the hGH-XTEN fusion protein is effective against the development of indicia of the disease or condition.

In one additional aspect, the hGH-XTEN fusion proteins provide an ameliorative effect against the development of, or the progression of, clinical and/or histological and/or biochemical and/or pathological indicia (including both symptoms and signs) of diseases or conditions related to AGHD in a human subject. In one embodiment, the indicia include an increased level of body fat (especially central or trunk adiposity, i.e., the waist), anxiety and depression, lethargy, changes in mood, feelings of isolation from others, a lack of motivation, elevated levels of cholesterol in the blood (e.g., abnormally high levels of low-density lipoproteins when compared to high density lipoproteins), elevated levels of triglycerides in the blood, decreased sexual function and interest, fatigue, decreased lean muscle mass, decreased extracellular fluid volume, decreased muscle strength, decreased physical energy and stamina, and reduced bone density. In another embodiment, the subject is at risk for a disease of condition related to AGHD. In general, a subject at risk will previously have incurred some damage to the pituitary gland and/or the hypothalamus. In one embodiment, the subject at risk was previously diagnosed as having a tumor associated with the pituitary gland, and/or underwent surgery, chemotherapy, or radiation therapy to treat the tumor. In another embodiment, the subject at risk previously had or presently has a reduced blood supply to the pituitary gland. In one other embodiment, the subject at risk previously suffered cranial ablation or has a history of head trauma. In some embodiments, the subject at risk previously or presently suffers from a hypothalamic-pituitary disease or disorder.

The efficacy of the treatment of diseases and conditions described herein (including GHD) can be measured by various assessments commonly used in evaluating GHD. For example, the health of hormone-secreting glands can be evaluated by, but not limited to, e.g., IGF-I standard deviation score (SDS), growth hormone stimulation test (GHST), growth hormone releasing hormone (GHRH), stimulation tests, monitoring or measurement of endogenous hGH pulses, IGF-I levels, IGF-I binding protein levels, other blood or biochemical tests (e.g., total cholesterol, low-density lipoprotein (LDL) cholesterol, high-density lipoprotein (HDL) cholesterol, triglyceride, and lipids).

### VII). Dosage Forms and Pharmaceutical Compositions

In another aspect, the present invention provides doses or dosage forms comprising an hGH-XTEN fusion protein described herein.

In one embodiment, the dose or dosage of an hGH-XTEN fusion protein comprises a therapeutically effective dose for a human patient. In one other embodiment, the dose or dosage comprises between about 5 mg/administration and about 500 mg/administration of hGH-XTEN fusion protein.

In one other embodiment, the dose or dosage of hGH-XTEN fusion protein is selected from the group consisting of about 5 mg, about 6 mg, about 8 mg, about 10 mg, about 12 mg, about 14 mg, about 16 mg, about 18 mg, about 20 mg, about 24 mg, about 28 mg, about 30 mg, about 32 mg, about 36 mg, about 40 mg, about 44 mg, about 48 mg, about 50 mg, about 52 mg, about 56 mg, about 60 mg, about 64 mg, about 68 mg, about 70 mg, about 72 mg, about 76 mg, about 80 mg, about 82 mg, about 88 mg, about 90 mg, about 92 mg, about 96 mg, about 100 mg, about 104 mg, about 108 mg, about 110 mg, about 112 mg, about 116 mg, about 120 mg, about 124 mg, about 128 mg, about 130 mg, about 132 mg, about 136 mg, about 140 mg, about 144 mg, about 148 mg, about 150 mg, about 152 mg, about 156 mg, about 160 mg, about 164 mg, about 168 mg, about 170 mg, about 172 mg, about 176 mg, about 180 mg, about 184 mg, about 188 mg, about 190 mg, about 192 mg, about 196 mg, about 200 mg, about 204 mg, about 208 mg, about 210 mg, about 212 mg, about 216 mg, about 220 mg, about 224 mg, about 228 mg, about 230 mg, about 232 mg, about 236 mg, about 240 mg, about 244, about 248 mg, about 250 mg.

In other embodiments, the dose or dosage is (i) for use in treating human AGHD in a subject in need, e.g., a human patient; and/or (ii) formulated for subcutaneous administration. In one other embodiment, the hGH-XTEN fusion protein comprises the amino acid sequence shown as set forth in FIG. 1 (SEQ ID NO:1). In one embodiment, the dose or dosage form is a pharmaceutical composition comprising the fusion protein having the sequence as set forth in FIG. 1 (SEQ ID NO:1) and a pharmaceutically acceptable carrier.

In another embodiment, the invention provides kits, comprising packaging material and at least a first container comprising the pharmaceutical composition of the foregoing embodiment and a label identifying the pharmaceutical composition and storage and handling conditions, and a sheet of instructions for the preparation and/or administration of the pharmaceutical compositions to a subject.

In one additional aspect, the present invention provides compositions, pharmaceutical compositions, and dose amounts of an hGH-XTEN fusion protein. In some embodiments, the pharmaceutical composition or dose amount comprises a fusion protein having the sequence as set forth in FIG. 1 (SEQ ID NO:1), or a sequence having at least about 90% sequence identity to the sequence of SEQ ID NO.1. In some embodiments, the pharmaceutical composition or dose amount comprises a hGH-XTEN fusion protein comprising a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In some embodiments, the dose amount is for a human patient is not based upon the body weight or age of the patient. In some embodiments, the hGH-XTEN fusion protein is provided in the pharmaceutical composition, composition, or dose amount as a certain quantity. In some embodiments, the pharmaceutical composition or dose amount further comprises a pharmaceutically acceptable carrier.

It should be noted that where reference is made to a composition, pharmaceutical composition or dose amount comprising an amount of hGH-XTEN fusion protein between about a first mg and about a second mg, the “first mg” term may include the first mg value and the “second mg” term may include the second mg value.

### VIII). Articles of Manufacture

In one aspect, the present invention also provides kits and articles of manufacture containing materials useful for the treatment, prevention and/or diagnosis of disease (e.g., GHD). In another embodiment, the invention provides kits, comprising packaging material and at least a first container comprising a dosage form or pharmaceutical composition of the foregoing embodiment and a label identifying the dosage form or pharmaceutical composition and storage and handling conditions, and a sheet of instructions for the reconstitution and/or administration of the dosage form or pharmaceutical compositions to a subject. In one other embodiment, the kit includes a container and a label, which can be located on the container or associated with the container. The container may be a bottle, vial, syringe, cartridge (including auto injector cartridges), or any other suitable container, and may be formed from various materials, such as glass or plastic. The container holds a composition having an hGH-XTEN fusion protein as described herein, and may have a sterile access port. Examples of containers include a vial with a stopper that can be pierced by a hypodermic injection needle. The kits may have additional containers that hold various reagents, e.g., diluents, preservatives, and buffers. The label may provide a description of the composition as well as instructions for the intended use.

In one other aspect, the container is a pre-filled syringe. In one embodiment, the syringe is pre-filled with a composition having an hGH-XTEN fusion protein as described herein. In one additional aspect, the present invention provides containers of the composition having a hGH-XTEN fusion protein as described herein, wherein the container is suitable for autoinjection of the composition. In one embodiment, the container is a cartridge. In another embodiment, the container is a cartridge in an autoinjection pen. Those of ordinary skill in the art will appreciate that other suitable autoinjection devices may be used for the present invention. In some embodiments, the autoinjection device comprises a spring-loaded syringe within a cylindrical housing that shields the needle tip prior to injection. In one embodiment, the patient depresses a button on the device and the syringe needle is automatically inserted to deliver the contents.

In another embodiment, the device is a gas jet autoinjection device. In other embodiments, the gas jet device comprises a cylinder of pressurized gas but the needle is absent. Upon activation, the device propels a fine jet of liquid through the skin without the use of a needle. In one other embodiment, the device is an iontophoresis device or electromotive drug administration (EMDA) device (e.g., use of a small electric charge to deliver an agent through the skin without the use of a needle).

The kit has at least one container that includes a molecule comprising an hGH-XTEN fusion protein described herein as the active agent. The container may comprise an hGH-XTEN fusion protein dosage form or pharmaceutical composition. A label may be provided indicating that the dosage form or composition may be used to treat a disease. The label may also provide instructions for administration to a subject in need of treatment. The kit may further contain an additional container having a pharmaceutically-acceptable buffer, such as bacteriostatic water for injection (BWFI), phosphate-buffered saline, Ringer's solution and dextrose solution. Finally, the kit may also contain any other suitable materials, including other buffers, diluents, filters, needles, and syringes.

In one aspect, the present invention provides a kit comprising a container which holds a pharmaceutical composition for administration to a human patient comprising a hGH-XTEN fusion protein. In one embodiment, the hGH-XTEN fusion protein comprises an amino acid sequence having at least about 90% sequence identity to the sequence set forth in FIG. 1 (SEQ ID NO.1). In one embodiment, the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, the N-terminus XTEN domain of SEQ ID NO: 1, and any of the XTEN domains from Table 1. In another embodiment, the kit further comprises a package insert associated with said container. In one other embodiment, the package insert indicates that said composition is for the treatment of growth hormone deficiency by administration of more than one dose of the composition. In one embodiment, the administration is an administration of an initial dose of between about 5 mg/administration and about 500 mg/administration of the hGH-XTEN and a plurality of subsequent doses of the hGH-XTEN in an amount of between about 5 mg/administration and about 500 mg/administration. In another embodiment, the doses are separated in time from each other by at least about 15±2 days. The package insert may further indicate different doses, dose ranges, and times between doses as described herein. In one additional embodiment, the human patient is an adult. In some embodiments, the container is a pre-filled syringe or an autoinjection pen.

The following are non-limiting examples of methods, treatment regimens, and compositions of the invention. It is understood that various other embodiments may be practiced, given the general description provided above. Some aspects and embodiments of the compositions, kits, apparatuses and methods are set forth in the following separately numbered paragraphs.

1. A method of treating human adult growth hormone deficiency (AGHD) in an adult patient, the treatment comprising administering to the adult patient with AGHD a human growth hormone-XTEN (hGH-XTEN) fusion protein at a dose between about 5 mg/administration and about 500 mg/administration, wherein the dose is not based on a body weight of said adult patient.

2. The method of paragraph 1, wherein the administering is at a frequency of twice a month (15 days+/−2 days).

3. The method of paragraphs 1 or 2, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, and the N-terminus XTEN domain of SEQ ID NO: 1.

4. The method of paragraphs 1 to 3, wherein the treatment continues for at least about 6 months from first administration.

5. The method of paragraphs 1 to 3, wherein the treatment continues for at least about 12 months from first administration.

6. The method of paragraphs 1 to 5, wherein the first and the second XTEN are sequences that exhibit at least about 90% sequence identity to a sequence selected from the C-terminus XTEN domain of SEQ ID NO: 1, and the N-terminus XTEN domain of SEQ ID NO: 1.

7. The method of any one of paragraphs 1 to 6, wherein the dose of the hGH-XTEN fusion protein is administered subcutaneously.

8. The method of any one of paragraphs 1 to 7, wherein the dose of hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration.

9. The method of any one of paragraphs 1 to 8, wherein the patient has a serum IGF-I standard deviation score (SDS) between about −2.0 and about 2.0 following administration.

10. The method of paragraph 9, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5.

11. The method of any one of paragraphs 1 to 10, wherein the hGH-XTEN fusion protein comprises the amino acid sequence of SEQ ID NO:1.

12. The method of any one of paragraphs 1 to 11, wherein dose is effective to maintain a plasma concentration of said fusion protein in the patient at more than about 10 ng/mL for a period of at least 10 days after administration.

13. The method of any one of paragraphs 1 to 12, wherein the AGHD patient is a woman on estrogen therapy and the dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration.

14. The method of any one of paragraphs 1 to 12, wherein the AGHD patient is not a woman on estrogen therapy and the dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration.

15. The method of any one of paragraphs 1 to 12, wherein the AGHD patient is a patient having a baseline IGF-I SDS of less or equal than −1 and the dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration.

16. The method of any one of paragraphs 1 to 12, wherein the AGHD patient is a patient having a baseline IGF-I SDS of higher than −1 and the dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

17. A method of treating AGHD in an adult patient, the treatment comprising:
(a) administering to the adult patient with AGHD a hGH-XTEN fusion protein at a dose selected from (i) 20 mg for all subjects other than women receiving oral estrogen, or (ii) 40 mg for women receiving oral estrogen;
(b) monitoring the IGF-I standard deviation score (SDS) in a plasma or serum sample obtained from the patient during an initial dosage period of administration of an initial dose of hGH-XTEN fusion protein; and
(c) determining a subsequent dose of hGH-XTEN fusion protein administered over a subsequent dosage period based on the IGF-I SDS observed during the initial dosage period;
   wherein the dose is not based on a body weight of said adult patient.

18. The method of paragraph 17, wherein the wherein the administering is at a frequency of twice a month (15 days+/−2 days).

19. The method of paragraphs 17 or 18, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, and the N-terminus XTEN domain of SEQ ID NO: 1.

20. The method of paragraphs 17 to 19, wherein the hGH-XTEN fusion protein comprises the amino acid sequence of SEQ ID NO:1.

21. The method of paragraphs any one of paragraphs 17 to 20, comprising administering a plurality of subsequent doses between 5 mg/administration and 500 mg/administration until a maintenance dose is achieved.

22. The method of paragraph 21, wherein the maintenance dose is effective to maintain the patient's serum IGF-I standard deviation score (SDS) between −2.0 and 2.0 SDS for 7 days after two consecutive subsequent dose administrations.

23. The method of paragraph 22, wherein the maintenance dose is effective to maintain the patient's serum IGF-I standard deviation score (SDS) between −1.5 and 2.0 SDS for 7 days after two consecutive subsequent dose administrations.

24. The method of paragraph 22, wherein the maintenance dose is effective to maintain the patient's serum IGF-I standard deviation score (SDS) between 0 and 2.0 SDS for 7 days after two consecutive subsequent dose administrations.

25. The method of any one of paragraphs 17 to 24, wherein the dose of the hGH-XTEN fusion protein is administered subcutaneously.

26. The method of any one of paragraphs 21 to 24, wherein the dose of hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration.

27. The method of any one of paragraphs 21 to 24, wherein the maintenance dose is effective to maintain a plasma concentration of said fusion protein in the patient at more than about 10 ng/mL for a period of at least 10 days after administration.

28. The method of any one of paragraphs 17 to 27, wherein the AGHD patient is a woman on estrogen therapy and the dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration.

29. The method of any one of paragraphs 17 to 27, wherein the AGHD patient is not receiving oral estrogen and the dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration.

30. The method of any one of paragraphs 17 to 27, wherein the AGHD patient is a patient having a baseline IGF-I SDS of less or equal than −1 and the dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration.

31. The method of any one of paragraphs 17 to 27, wherein the AGHD patient is a patient having a baseline IGF-I SDS of higher than −1 and the dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

32. A method for the treatment of AGHD in an adult patient comprising: administering to an adult with AGHD a hGH-XTEN fusion protein at an initial dose between about 5 mg/administration and about 500 mg/administration, and as a plurality of subsequent doses between about 5 mg/administration and about 500 mg/administration, wherein the initial dose and the plurality of subsequent doses are not based on a body weight of said adult patient.

33. The method of paragraph 32, wherein the administering is at a frequency of twice a month (15 days+/−2 days).

34. The method of paragraphs 32 or 33, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, and the N-terminus XTEN domain of SEQ ID NO: 1.

35. The method of paragraphs 32 to 34, wherein said plurality of subsequent doses are effective to maintain the patient's serum IGF-I standard deviation score (SDS) between −2.0 and 2.0 SDS for 7 days after administration.

36. The method of paragraph 35, wherein said plurality of subsequent doses are effective to maintain the patient's serum IGF-I standard deviation score (SDS) between −1.5 and 2.0 SDS for 7 days after administration.

37. The method of paragraph 36, wherein said plurality of subsequent doses are effective to maintain the patient's serum IGF-I standard deviation score (SDS) between 0 and 2.0 SDS for 7 days after administration.

38. The method of any one of paragraphs 32 to 37, wherein the dose of the hGH-XTEN fusion protein is administered subcutaneously.

39. The method of any one of paragraphs 32 to 38, wherein either the initial dose or any of said plurality of doses of said hGH-XTEN fusion protein is between about 10 mg/administration and about 400 mg/administration, or between 20 mg/administration and about 400 mg/administration, or between 20 mg/administration and about 300 mg/administration, or between 20 mg/administration and about 250 mg/administration.

40. The method of any one of paragraphs 32 to 39, wherein said plurality of subsequent doses are effective to maintain a plasma concentration of said fusion protein in the patient at more than about 10 ng/mL for a period of at least 10 days after administration.

41. The method of any one of paragraphs 32 to 40, wherein the AGHD patient is a woman on estrogen therapy, and wherein either the initial dose or any of the plurality of doses of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration.

42. The method of any one of paragraphs 32 to 40, wherein the AGHD patient is not a woman on estrogen therapy, and wherein either the initial dose or any of the plurality of doses of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration.

43. The method of any one of paragraphs 32 to 40, wherein the AGHD patient is a patient having a baseline IGF-I SDS of less or equal than −1, and wherein either the initial dose or any of the plurality of doses of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration.

44. The method of any one of paragraphs 32 to 40, wherein the AGHD patient is a patient having a baseline IGF-I SDS of higher than −1, and wherein either the initial dose or any of the plurality of doses of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

45. A method of treating AGHD in an adult patient, the treatment comprising:
(a) administering to the adult patient with AGHD an hGH-XTEN fusion protein at an initial dose selected from (i) 20 mg for all subjects other than women receiving oral estrogen, or (ii) 40 mg for women receiving oral estrogen;
(b) decreasing or increasing one or more subsequent dosage amounts of the hGH-XTEN fusion protein until a maintenance dose is achieved, wherein said maintenance dose is achieved when the patient's serum IGF-I SDS is between about −2.0 and about 2.0 for two consecutive 7 post-dose days after administration;
(c) administering a plurality of subsequent maintenance doses;
   wherein the initial dose, the one or more subsequent doses, and the plurality of subsequent maintenance doses are not based on a body weight of said adult patient.

46. The method of paragraph 45, wherein the administering is at frequency of twice a month (15 days+/−2 days).

47. The method of paragraphs 45 to 46, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, and the N-terminus XTEN domain of SEQ ID NO: 1.

48. The method of paragraphs 45 to 47, wherein said maintenance dose is effective to maintain the patient's serum IGF-I SDS between about −2.0 and about 2.0 for (i) at least 7 days; (ii) at least about 10 days; or (iii) at least about 20 days after administration.

49. The method of paragraphs 45 to 48, wherein the hGH-XTEN fusion protein comprises the amino acid sequence of SEQ ID NO:1.

50. The method of any one of paragraphs 45 to 49, wherein the AGHD patient is a woman on estrogen therapy and the maintenance dose of the hGH-XTEN fusion protein is between about 30 mg/administration and about 250 mg/administration, or between about 35 mg/administration and about 250 mg/administration, or between about 40 mg/administration and about 250 mg/administration, or between about 45 mg/administration and about 250 mg/administration; or between about 30 mg/administration and about 250 mg/administration, or between about 5 mg/administration and about 250 mg/administration.

51. The method of any one of paragraphs 45 to 49, wherein the AGHD patient is not receiving oral estrogen and the maintenance dose of the hGH-XTEN fusion protein is between about 10 mg/administration and about 250 mg/administration, or between about 10 mg/administration and about 200 mg/administration, or between about 10 mg/administration and about 150 mg/administration, or between about 10 mg/administration and about 100 mg/administration, or between about 10 mg/administration and about 90 mg/administration, or between about 10 mg/administration and about 80 mg/administration, or between about 10 mg/administration and about 60 mg/administration, or between about 10 mg/administration and about 50 mg/administration, or between about 10 mg/administration and about 40 mg/administration.

52. The method of any one of paragraphs 45 to 49, wherein the AGHD patient is a patient having a baseline IGF-I SDS of less or equal than −1 and the maintenance dose of the hGH-XTEN fusion protein is between about 10 mg/administration and about 250 mg/administration, or between about 10 mg/administration and about 150 mg/administration, or between about 10 mg/administration and about 80 mg/administration, or between about 20 mg/administration and about 80 mg/administration.

53. The method of any one of paragraphs 45 to 49, wherein the AGHD patient is a patient having a baseline IGF-I SDS of higher than −1 and the maintenance dose of the hGH-XTEN fusion protein is 20 mg/administration and about 60 mg/administration, or between about 20 mg/administration and about 50 mg/administration, or between about 20 mg/administration and about 40 mg/administration; or between about 20 mg/administration and about 30 mg/administration, or between about 10 mg/administration and about 40 mg/administration, or between about 10 mg/administration and about 30 mg/administration.

### EXAMPLES

### Example 1: Lone-Term Extension Study of the Safety of VRS-317 in Adults with Growth Hormone Deficiency (GHD)

Adults age 18 and over who have well-established growth hormone deficiency (GHD), according to the Endocrine Society guidelines, receive twice-monthly (every 15 days±2 days) subcutaneous (SC) doses of VRS-317 (SEQ ID NO: 1). Doses are titrated to each subject's individual IGF-I responses based on the IGF-I level 7 days post-dose until a maintenance dose is achieved. In this study, a maintenance dose is defined as an IGF-I value between 0 and 2.0 SDS for two consecutive 7 day post-dose time points (Day 8, peak level). Subjects enrolling in this study are assigned to one of two cohorts and receive a starting dose of 20 mg twice-monthly (40 mg for women on estrogen therapy) (Table 1) and are titrated per the Dose Titration Plan (Table 2). Maintenance doses may be adjusted further based on pharmacodynamics (PD) data at the discretion of the Investigator or Medical Monitor while maintaining IGF-I at Day 8 (7 days post dose) within target range.

For patients on maintenance dose that have IGF-I levels >2.0 SDS at any point during the maintenance period, dose adjustment is made by the Investigator or Medical Monitor based on clinical judgment with the objective to achieve and maintain IGF-I at day 7 post-dose between 0 and 2.0 SDS. Any change in VRS-317 dose in a subject that has achieved maintenance dosing is followed by an ad-hoc Day 8 IGF-I measurement to ensure IGF-I values are within target range (0 and 2.0 SDS).

TABLE 1
Study Drug Starting Dose Information:

| | Fixed Starting | Frequency of |
|---|---|---|
| Subject Cohort Assignment | Study Dose | Administration |
| Cohort 1: Adults with GHD | 20 mg | Twice-Monthly |
| irrespective of age and gender | | |
| Cohort 2: Women receiving oral | 40 mg | Twice-Monthly |
| estrogens | | |

TABLE 2
Dose Titration Plan

| | | |
|---|---|---|
| IGF-I at Day 8 | Cohort 1 | Cohort 2 |

Downward Dose Titration

| | | |
|---|---|---|
| 0 to 2.0 SDS | No change in dosing | No change in dosing |
| 2.01 to 3.0 SDS | 4 mg dose decrement | 4 mg dose decrement |
| Greater than 3.0 SDS | 10 mg dose decrement | 10 mg dose decrement |

Upward Dose Titration

| | | |
|---|---|---|
| −1.0 to 0 SDS | 4 mg dose increment | 10 mg dose increment |
| Less than −1.0 | 10 mg dose increment | 20 mg dose increment |
| to −2.0 SDS | | |
| Less than −2.0 SDS | 20 mg dose increment | 40 mg dose increment |

Doses are scheduled relative to Day 1, not relative to the prior dose, and are administered every 15 days±2 days, unless a dose outside of this time window is approved by the Medical Monitor.

For subjects, treatment is initiated at the fixed starting dose (20 mg or 40 mg, as per assigned cohort), with subsequent doses determined based on individual IGF-I response. IGF-I levels (ng/mL) are checked at pre-dose (trough) and at 7 (±1) days after dosing (Day 8, peak) until dose stabilization is achieved. The 7 day post-dose (Day 8, peak) IGF-I SDS value is used to determine dose adjustments with a target range of 0 to 2.0 SDS. If the peak IGF-I SDS is below the target range (e.g., <0 SDS), the dose is titrated up according to the Dose Titration Plan (Table 2). If the peak IGF-I SDS is above the target range (e.g., >2.0 SDS), the dose is titrated down according to the Dose Titration Plan (Table 2). Once a subject has achieved an IGF-I SDS within the target range for two consecutive months, s/he is considered dose stabilized and continues to receive that maintenance dose for the remainder of the study unless further adjustment becomes necessary. After dose stabilization is achieved, IGF-I levels are no longer tested monthly; maintenance subjects have pre-dose and 7 days post-dose IGF-I levels drawn every 3 months for the first year, and then every 6 months thereafter. If a subject on maintenance dose has an IGF-I value outside the target range, further dose adjustments are considered on an individual basis by the Investigator or Medical Monitor with the objective to achieve and maintain IGF-I SDS at Day 8 within target range (IGF-I SDS 0-2.0). Any change in VRS-317 dose in a subject that has achieved maintenance dosing is followed by an ad-hoc Day 8 IGF-I measurement to ensure IGF-I values are within target range (0 and 2.0 SDS).

Pharmacokinetics (PK) and Pharmacodynamics (PD)

The PK parameter is VRS-317 concentration and the PD parameters are serum IGF-I and IGFBP-3. All subjects have blood samples collected for PK/PD assessments at Day 1 (pre-dose) and Days 8, 16, and 23 during Month 1 and at Day 1 (pre-dose) and at Day 8 monthly until dose stabilization is achieved. Once maintenance dosing is achieved, PK/PD samples are collected quarterly at Day 1 and Day 8 in the first year and then every 6 months thereafter.

Safety

Subjects are monitored for safety throughout their participation in the study. Safety is monitored by physical examination, inspection of injection sites, vital signs, ECGs, and clinical laboratory determinations. Adverse events (AEs) and concomitant medications (CMs) are captured. AEs are graded using the Common Terminology Criteria for Adverse Events (CTCAE). AEs are coded using the MedDRA dictionary and CMs using the WHO Drug Dictionary (WHODDE, WHO Drug Dictionary Enhanced).

An external data and safety monitoring board (DSMB) monitor and protect the safety of the study subjects throughout the study duration. DSMB meetings occur twice yearly (approximately every 6 months).

Test Product, Dose, and Mode of Administration

VRS-317 is administered as subcutaneous injection(s) in the thigh, abdomen, upper arm or buttocks. Administration of injections is rotated to different injection sites. Study drug is administered by trained subjects/caregivers, or a health care professional. A subject is considered to have achieved a maintenance stable dose when two consecutive 7 days post-dose (Day 8, peak) IGF-I SDS values are between 0 and 2.0 SDS. Subsequently, the subject continues to receive the maintenance dose twice-monthly (every 15 days±2 days). Maintenance doses are adjusted further at the discretion of the Investigator or Medical Monitor while keeping IGF-I at 7 days post-dose between 0 and 2.0 SDS.

If a subject on a maintenance dose has an IGF-I SDS value above 2.0 SDS, dose adjustments are made by the Investigator or Medical Monitor based on clinical judgment with the objective to achieve and maintain IGF-I at day 7 post-dose between 0 and 2.0 SDS.

Adjustments are implemented to alleviate safety concerns or optimize normalization of IGF-I SDS responses. In some cases, the Investigator and Medical Monitor may agree to temporarily alter or suspend a subject's dose during illness or injury. If a subject on maintenance dose has an IGF-I value outside the target range, further dose adjustments is considered on an individual basis by the Investigator or Medical Monitor with the objective to achieve and maintain IGF-I SDS at Day 8 within target range (IGF-I SDS between 0-2.0)

Statistical Methods

Safety data is listed by subject and summarized by cohort using frequencies or descriptive statistics. All subjects who receive at least one dose of VRS-317 are included in the safety analysis. Summaries of all adverse events (AEs), serious adverse events (SAEs), and incidence of Grade 3 or 4 adverse events are be classified according to severity and relationship to study drug.

For continuous variables, descriptive statistics include the number of subjects, mean, standard deviation (SD), median, minimum, and maximum. For categorical variables, descriptive statistics include the number and percentage of subjects in each category. Summaries of subject disposition, demographics, disease characteristics, and exposure and response to dosing of study medication are produced.

### Example 2: Results of Twice-Monthly Administration of Individualized Doses of VRS-317

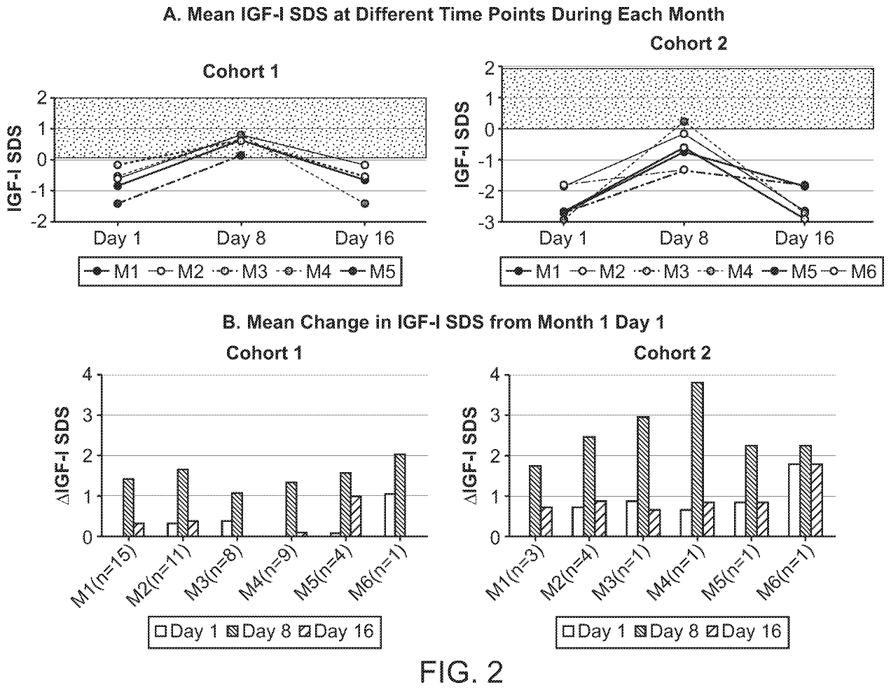

A good IGF-I response to twice-monthly somavaratan was observed with repeat dosing involving dose titrations. By Day 8, the mean IGF-I SDS increased between 1 and 3.8 SDS from the Month 1 pre-dose IGF-I SDS. Before each dose, IGF-I returned to values similar to or slightly higher than the Month 1 pre-dose, as seen in FIG. 2.

In these analyses, nine of 19 subjects (47.4%; all in Cohort 1) exposed to twice-monthly somavaratan achieved a maintenance dose (8 at Month 2 without dose adjustment, and one at Month 4 after three dose adjustments). The mean±standard deviation (SD) maintenance dose was 28.72±12.1 mg twice monthly.

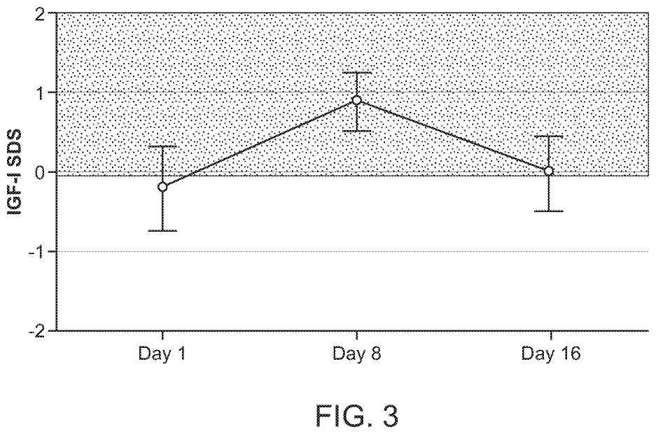

The mean±SD IGF-I SDS during the dosing interval for the nine subjects on a maintenance dose is shown in FIG. 3.

A good IGF-I response was observed in this group of patients, with a mean change from baseline (pre-dose Month 1) to Day 8 between 0.85-1.53 SDS. IGF-I SDS by Day 16 was slightly higher than baseline. IGF-I did not exceed 2 SDS at any point.

Neither the pre-dose IGF-I nor the Day 8 IGF-I SDS appeared to increase over time, indicating no drug accumulation with twice-monthly somavaratan administration.

The mean±SD somavaratan doses in this group of patients are 23.5±7.1 mg (range 16-40 mg) and 144.2±95.4 mg (range 45-247.7 mg) for Cohorts 1 and 2, respectively.

In conclusion, the new dosing regimen, target IGF-I and dose titration plan appear to be appropriate to elicit a PD response, while preventing IGF-I excursions in most of the subjects and allowing for dose titration based on individual response.

1. A method of treating human adult growth hormone deficiency (AGHD) in an adult patient, the treatment comprising administering to the adult patient with AGHD a human growth hormone-XTEN (hGH-XTEN) fusion protein at a dose between about 5 mg/administration and about 500 mg/administration, wherein the dose is not based on a body weight of said adult patient.

2. The method of claim 1, wherein the administering is at a frequency of twice a month (15 days±2 days).

3. The method of claim 1, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 80% sequence identity to a sequence selected from the C-terminus XTEN domain of SEQ ID NO: 1, and the N-terminus XTEN domain of SEQ ID NO: 1.

4. The method of claim 1, wherein the treatment continues for at least about 6 months from first administration.

5. (canceled)

6. (canceled)

7. The method of claim 1, wherein the dose of the hGH-XTEN fusion protein is administered subcutaneously.

8. The method of claim 1, wherein the dose of hGH-XTEN fusion protein is between about 10 mg/administration and about 400 mg/administration.

9. The method of claim 1, wherein the patient has a serum IGF-I standard deviation score (SDS) between about −2.0 and about 2.0 following administration.

10. (canceled)

11. (canceled)

12. The method of claim 1, wherein the dose is effective to maintain a plasma concentration of said fusion protein in the patient at more than about 10 ng/mL for a period of at least 10 days after administration.

13. The method of claim 1, wherein the dose of the hGH-XTEN fusion protein is between about 5 mg/administration and about 250 mg/administration.

14. (canceled)

15. The method of claim 1, wherein the AGHD patient is a patient having a baseline IGF-I SDS of less or equal than −1 and the dose of the hGH-XTEN fusion protein is between about 10 mg/administration and about 250 mg/administration.

16. The method of claim 1, wherein the AGHD patient is a patient having a baseline IGF-I SDS of higher than −1 and the dose of the hGH-XTEN fusion protein is between 20 mg/administration and about 60 mg/administration, or between about 10 mg/administration and about 40 mg/administration.

17-31. (canceled)

32. A method for the treatment of AGHD in an adult patient comprising: administering to an adult with AGHD a hGH-XTEN fusion protein at an initial dose between about 5 mg/administration and about 500 mg/administration, and as a plurality of subsequent doses between about 5 mg/administration and about 500 mg/administration, wherein the initial dose and the plurality of subsequent doses are not based on a body weight of said adult patient.

33. (canceled)

34. (canceled)

35. The method of claim 32, wherein said plurality of subsequent doses are effective to maintain the patient's serum IGF-I standard deviation score (SDS) between −2.0 and 2.0 SDS for 7 days after administration.

36. (canceled)

37. (canceled)

38. (canceled)

39. The method of claim 32, wherein either the initial dose or any of said plurality of doses of said hGH-XTEN fusion protein is between about 10 mg/administration and about 400 mg/administration.

40. The method of claim 32, wherein said plurality of subsequent doses are effective to maintain a plasma concentration of said fusion protein in the patient at more than about 10 ng/mL for a period of at least 10 days after administration.

41. The method of claim 32, wherein either the initial dose or any of the plurality of doses of the hGH-XTEN fusion protein is between about 5 mg/administration and about 250 mg/administration.

42. (canceled)

43. The method of claim 32, wherein the AGHD patient is a patient having a baseline IGF-I SDS of less or equal than −1, and wherein either the initial dose or any of the plurality of doses of the hGH-XTEN fusion protein is between about 10 mg/administration and about 250 mg/administration.

44. The method of claim 32, wherein the AGHD patient is a patient having a baseline IGF-I SDS of higher than −1, and wherein either the initial dose or any of the plurality of doses of the hGH-XTEN fusion protein is between about 20 mg/administration and about 60 mg/administration, between about 10 mg/administration and about 40 mg/administration.

45. A method of treating AGHD in an adult patient, the treatment comprising:
(a) administering to the adult patient with AGHD an hGH-XTEN fusion protein at an initial dose selected from (i) 20 mg for all subjects other than women receiving oral estrogen, or (ii) 40 mg for women receiving oral estrogen;
(b) decreasing or increasing one or more subsequent dosage amounts of the hGH-XTEN fusion protein until a maintenance dose is achieved, wherein said maintenance dose is achieved when the patient's serum IGF-I SDS is between about −2.0 and about 2.0 for two consecutive 7 post-dose days after administration;
(c) administering a plurality of subsequent maintenance doses;
wherein the initial dose, the one or more subsequent doses, and the plurality of subsequent maintenance doses are not based on a body weight of said adult patient.

46. The method of claim 45, wherein the administering is at frequency of twice a month (15 days±2 days).

47. The method of claim 45, wherein the hGH-XTEN fusion protein comprises a single GH molecule linked to a first and a second XTEN, with an N- to C-terminus configuration of XTEN-GH-XTEN, in which the GH is a sequence that exhibits at least about 90% sequence identity to the human growth hormone protein sequence (SEQ ID NO: 2), and the first and the second XTEN are sequences that exhibit at least about 90% sequence identity to a sequence selected from the C-terminus XTEN domain of SEQ ID NO: 1, and the N-terminus XTEN domain of SEQ ID NO: 1.

48. The method of claim 45, wherein said maintenance dose is effective to maintain the patient's serum IGF-I SDS between about −2.0 and about 2.0 for (i) at least 7 days; (ii) at least about 10 days; or (iii) at least about 20 days after administration.

49. (canceled)

50. The method of claim 45, wherein the maintenance dose of the hGH-XTEN fusion protein is between about 5 mg/administration and about 250 mg/administration.

51. (canceled)

52. The method of claim 45, wherein the AGHD patient is a patient having a baseline IGF-I SDS of less or equal than −1 and the maintenance dose of the hGH-XTEN fusion protein is between about 10 mg/administration and about 250 mg/administration.

53. The method of claim 45, wherein the AGHD patient is a patient having a baseline IGF-I SDS of higher than −1 and the maintenance dose of the hGH-XTEN fusion protein is 20 mg/administration and about 60 mg/administration or between about 10 mg/administration and about 40 mg/administration.
